(19) United States
(12) Patent Application Publication
(10) Pub. No.: US 2007/0099233 A1
(43) Pub. Date: May 3, 2007

(54) Cross-reference

(76) Inventor: Jeffrey B. Stock, Rocky Hill, NJ (US)

Correspondence Address: PATENT DOCKET ADMINISTRATOR;LOWENSTEIN SANDLER PC, 65 LIVINGSTON AVENUE, ROSELAND, NJ, 07068, US

(21) Appl. No.: 10/561,828

(22) PCT Filed: Jun. 24, 2004
(86) PCT No.: PCT/US04/20529

§ 371 Date: Dec. 22, 2005

Related U.S. Application Data
(60) Provisional application No. 60/483,011, filed on Jun. 27, 2003.

(51) Int. Cl. G01N 33/53; C12M 1/34

(52) U.S. Cl. 435/7.1; 435/287.2

(57) ABSTRACT

The invention provides sensors that are useful for detecting and measuring chemical characteristics and physical properties of compositions containing analytes. The invention further provides, among other things, methods for making the sensors and for using them, as well as modified cells having sensors of the invention therein.

### CROSS-REFERENCE

This application claims priority to U.S. Provisional Patent Application 60/483,011 filed Jun. 27, 2003, which is incorporated herein by reference in its entirety.

### BACKGROUND

Sensor technology is pervasive. Familiar mechanical deployments of sensors include, to name just a few, simple thermostats, humidity and temperature sensors in HVAC systems and appliances, sensors in engine control systems, sensors that trigger air bags to inflate, sensors in process control systems, infrared and motion sensors in security devices, smoke, CO, and natural gas sensors in safety warning devices, and photosensors in photosensitive light switches and automatic door openers.

Sensors also have been deployed in a variety of more specialized, perhaps less familiar applications, that include their use in medical monitoring equipment, in devices that control the delivery of fluids to patients, in sophisticated diagnostic assay systems used in clinical laboratories, and in complex instrumentation used in virtually every research endeavor and every technology development project.

In these, and in most other applications, present day sensors generally are limited to sensing reliably and accurately a single environmental variable, and are based on changes in a mechanical and/or electrical property of a material reliably induced by changes in the environmental variable of interest. Changes in temperature cause mechanical change (expansion and contraction) in the metals that make up the bimetallic coil of certain types of thermostat, for instance. In household smoke alarms, particles in smoke reduce the radiation reaching a collector and thereby change the electrical properties of the collector. When the change exceeds a preset threshold, the alarm goes off. More sophisticated sensors make similar use of laser beams, motion detectors, miniature accelerometers and the like, including those used in chemical process controllers.

The sensors in widespread use generally respond to physical phenomena: they are not chemosensors. Indeed, even sensors employed to monitor chemical process variations generally do so by monitoring a physical variable, such as color depth, reflectivity, or polarization. Clinical laboratory assay devices employed to recognize specific chemical species are a notable exception; but, clinical laboratory analyzers typically can determine only one species at a time, require considerable sample preparation, are too slow to be used for real-time monitoring and generally must be purpose designed (i.e., assays for each new ligand or other analyte must be developed essentially de novo).

Platforms for detecting thousands of different analytes all-at-once have been developed during the past decade, primarily for research. The platforms initially were limited to either DNA or peptides. More recently, arrays of compounds of other types have been developed as well, including arrays of proteins, arrays of inorganic compound libraries, arrays of small organic compound libraries, arrays of carbohydrate compounds, arrays of cells, arrays of tissue samples, arrays of aptamers, arrays of antibodies and arrays of other ligand binding substances.

While these platforms are proving useful in research applications, their promise as the front end building blocks of real time sensors to simultaneously monitor many variables, such as a variety of analytes in samples, is limited by several factors, including the time required to carry out assays using the devices, sensitivity to process parameters, severe limits on comparability of data between different protocols and platforms, and even between datasets from the same protocol on the same platform.

These types of sensors provide a quantitatively variable output signal that is directly related to the concentration of target analyte. Downstream detectors must interpret the output signal so that effector components can be directed to produce appropriate responses (e.g. the thermostat's output is coupled to a switching device to turn on or off a heater). Conventional sensors do not combine these modalities. Analyte concentrations are measured by distinct sensory devices whose outputs are fed into independent signal receivers that filter, amplify, and otherwise modulate the output information, which then is fed into an a controller that turns some effector apparatus on or off. Thus, the sensor and output cassettes are independent and unrelated entities, and there is no adaptation cassette to provide the sensor with an inherent intelligence with an ability to convert input information (analyte concentrations) into a suitable output signal (turn on or off one or more effector devices) within the context of a single entity.

There is a need, accordingly, for improved sensors that are more sensitive, quicker, produce results that can be compared across experiments, cost less, have improved analyte binding capabilities, that can be produced using routine techniques, provide a modular sensing and/or reporter system through which signaling modules readily can be operatively linked to a variety of sensing modules, and both sensing and signaling modules likewise can be operatively linked to a variety of modification modules, that function nearly in real time, or in real time, that is sensitive, that is economical, and that can be used to detect a great many analytes, preferably in parallel, inter alia.

Furthermore, there is a need for sensors in which the gain can be programmed and/or adjusts dynamically to changes in the environment. Importantly, there is a need for intelligent sensors that can be programmed to produce a signal in response to combinations of sensory information, particularly sensors that can be programmed to integrate desired ranges, patterns and combinations of sensory information. Further in this regard there is a need for intelligent sensors to directly control effector outputs. Particularly, there is a need for the same in all these regards that is a single nanoscale entity, or comprised of a few nanoscale components.

### SUMMARY OF THE INVENTION

One aspect of the invention is a sensor comprising a sensor element comprising at least one sensing moiety and at least one signaling moiety. Each sensing moiety has one or more states indicative of a target analyte. The signaling moiety (alone or in combination with another signaling moiety) engenders the production of a detectable signal. The state or a change in the state of the sensing moiety is transduced to one or more signaling moieties whereby the signaling moiety (alone or in combination with another) engenders production of a detectable signal qualitatively or quantitatively indicative of a condition. The condition may be, e.g., the absence below a threshold, the presence above a threshold, or the amount of a target analyte. The condition may be a change in the absence below a threshold, the presence above a threshold or the amount of a target analyte. Preferably one or the other or both the sensing moiety and the signaling moiety is not a naturally occurring entity.

Another aspect of the invention is a sensor comprised of a sensor element comprising a first plurality of sensing moieties and a second plurality of signaling moieties. Each sensing moiety has one or more states indicative of a target analyte. The state, or a change in the state, of one or more of the sensing moieties is transduced to one or more of signaling moieties. One signaling moiety alone or a third plurality of signaling moieties in combination engenders the production of a detectable signal qualitatively or quantitatively indicative of a condition, e.g. the absence below a threshold, the presence above a threshold, or the amount of a target analyte. Another condition may be a change in the absence below a threshold, the presence above a threshold, or the amount of a target analyte. Preferably the sensing moiety and the signaling moiety do not naturally occur with one another in the same organism.

Another aspect of the invention is a sensor comprised of sensor element comprising a first plurality of sensing moieties and a second plurality of signaling moieties. Each sensing moiety has one or more states indicative of a target analyte. The state, or a change in the state, of one or more of the sensing moieties is transduced to one or more of signaling moieties. One signaling moiety alone, or a third plurality of signaling moieties in combination, engenders the production of a detectable signal qualitatively or quantitatively indicative of a condition, e.g. the absence below a threshold, the presence above a threshold, or the amount of a target analyte. Alternatively, the condition may be a change in the absence below a threshold, the presence above a threshold, or the amount of a target analyte. The sensing moieties are comprised of one or more polypeptides, and one or more regions of the one or more polypeptides has a de novo polypeptide sequence.

Another aspect of the invention is a method for detecting a target analyte. The method comprises (1) exposing a composition in which the target analyte is to be detected to a sensor that produces a detectable signal indicative of the absence below a threshold; the presence above a threshold, the amount of the target analyte, or a change in the absence below a threshold, the presence above a threshold or the amount of the target analyte, and (2) determining the detectable signal or the absence thereof. The sensor is comprised of a sensor element comprising one or more sensing moieties and one or more signaling moieties. Each sensing moiety has one or more states indicative of a target analyte and each signaling moiety alone or in combination engenders the production of a detectable signal. The state or a change in the state of one or more sensing moieties is transduced to one or more signaling moieties whereby the one or more signaling moieties alone or in combination engender the production of a detectable signal qualitatively or quantitatively indicative of a condition. The condition may be (a) the absence below a threshold, the presence above a threshold or the amount of a target analyte or (b) a change in the absence below a threshold, the presence above a threshold or the amount of a target analyte. Preferably one or the other or both the sensing moiety and the signaling moiety is not a naturally occurring entity.

Another aspect of the invention is a method for detecting a target analyte. The method comprises (1) exposing a composition in which the target analyte is to be detected to a sensor that produces a detectable signal indicative of the absence below a threshold, the presence above a threshold or the amount of the analyte, or a change in the absence below a threshold, the presence above a threshold or the amount of the target analyte, and (2) determining the detectable signal or the absence thereof. The sensor is comprised of a sensor element comprising one or more sensing moieties and one or more sensing moieties, wherein each sensing moieties has one or more states indicative of a target analyte, each signaling moiety alone or in combination engenders the production of a detectable signal. The state or a change in the state of one or more sensing moieties is transduced to one or more signaling moieties whereby the one or more signaling moieties alone or in combination engender the production of a detectable signal qualitatively or quantitatively indicative of a condition. The condition may be the absence below a threshold, the presence above a threshold or the amount of a target analyte or of a change in said absence, presence, or the amount of the target analyte. The sensing moieties are comprised of one or more polypeptides, and one or more regions of one or more of the polypeptides has a de novo polypeptide sequence.

Another aspect of the invention is a modified bacterial cell. It comprises a first plurality of sensing moieties and a second plurality of signaling moieties. Each sensing moiety has one or more states indicative of a target analyte. The state, or a change in the state, of one or more of the signaling moieties is transduced to one or more of signaling moieties. One signaling moiety alone, or a third plurality of signaling moieties in combination, engenders the production of a detectable signal qualitatively or quantitatively indicative of a condition. The condition may be the absence below a threshold, the presence above a threshold, or the amount of a target analyte, or a change in said absence below, said presence above, or the amount of a target analyte. The sensing moieties are comprised of one or more polypeptides, and one or more regions of the polypeptides has a de novo polypeptide sequence.

A further aspect of the invention is a method for making a sensor, comprising preparing sensor elements from a bacterial cell culture. The sensor elements are operatively incorporated into a sensor. The sensor elements comprise a first plurality of sensing moieties and a second plurality of signaling moieties, wherein each sensing moiety has one or more states indicative of a target analyte. The state or a change in the state of one or more of the sensing moieties is transduced to one or more of signaling moieties. One signaling moiety alone, or a third plurality of signaling moieties in combination, engenders the production of a detectable signal qualitatively or quantitatively indicative of a condition, e.g., the absence below a threshold, the presence above a threshold, or the amount of a target analyte, or of change in said absence below, presence above, or the amount of a target analyte. One or more sensing moieties is comprised of one or more polypeptides, and one or more regions of one or more of the polypeptides has a de novo polypeptide sequence.

Other aspects will be apparent to one of ordinary skill upon reading the specification in detail.

### BRIEF DESCRIPTION OF THE FIGURES

FIG. 1 is a schematic depiction of a simple sensor in the presence of two concentrations of an analyte.

FIG. 2 is a schematic depiction of interchangeable sensing modules and a constant signaling module.

FIG. 3 is a schematic depiction of a sensor employing a pair of two state receptors that have the same sensing moiety with different signaling moieties.

FIG. 4 is a schematic diagram showing a sensor employing four two state receptors each specific for one of four different analytes.

FIG. 5 is a schematic depiction of a sensor employing four different two state receptors for a single analyte.

FIG. 6 is a schematic diagram depicting a sensor employing four different two state receptors, two specific to one analyte and two others specific to a second analyte.

FIG. 7 is a schematic depiction of a sensor employing a single five state receptor for a single analyte.

FIG. 8 is a schematic diagram showing a sensor employing four different five state receptors for four different analytes.

FIG. 9 is a schematic depiction of a sensor employing four different five-state receptors for a single analyte.

FIG. 10 is a schematic depiction of a sensor employing four different five-state receptors, two for one analyte and the other two for a different analyte.

FIG. 11 is a diagrammatic representation of a bacterial MCP and a MCP sensory apparatus: on the left is a single dimeric receptor unit and on the right is a membrane bound receptor multimer complexed with CheA and Chew.

FIG. 12 is a schematic depiction of the organization of a bacterial chemosensory organelle (above) and the general cellular location of the organelles (below).

FIG. 13 is a diagram showing a soluble receptor protein engineered from an E. coli MCP.

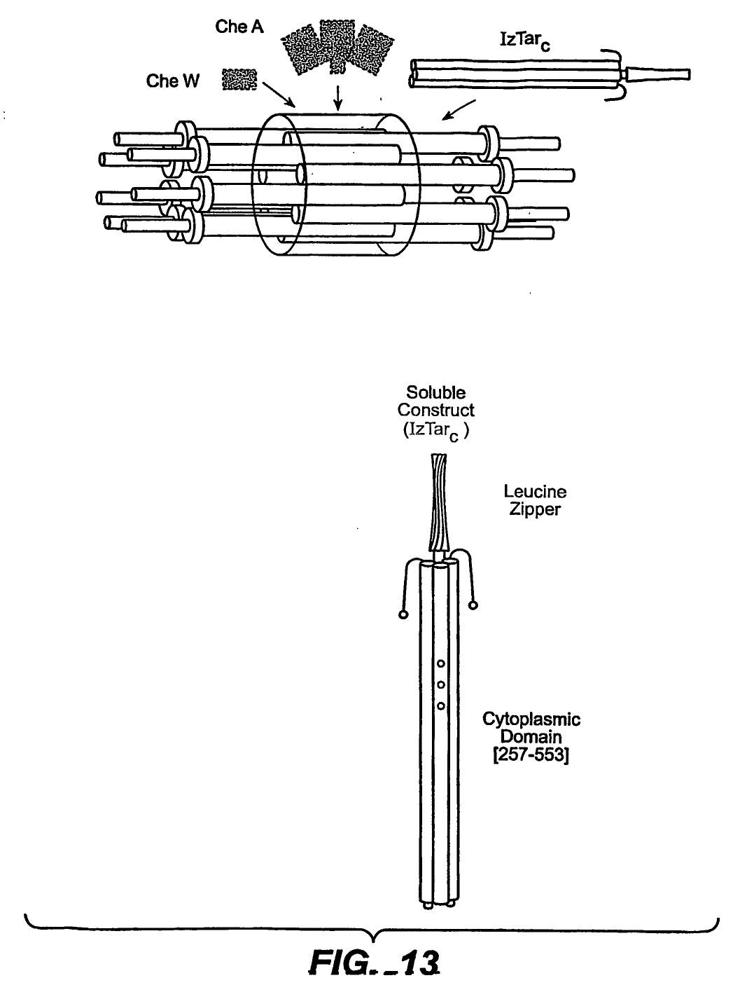

FIG. 14 is a schematic representation of components of adaptive bacterial chemosensory systems.

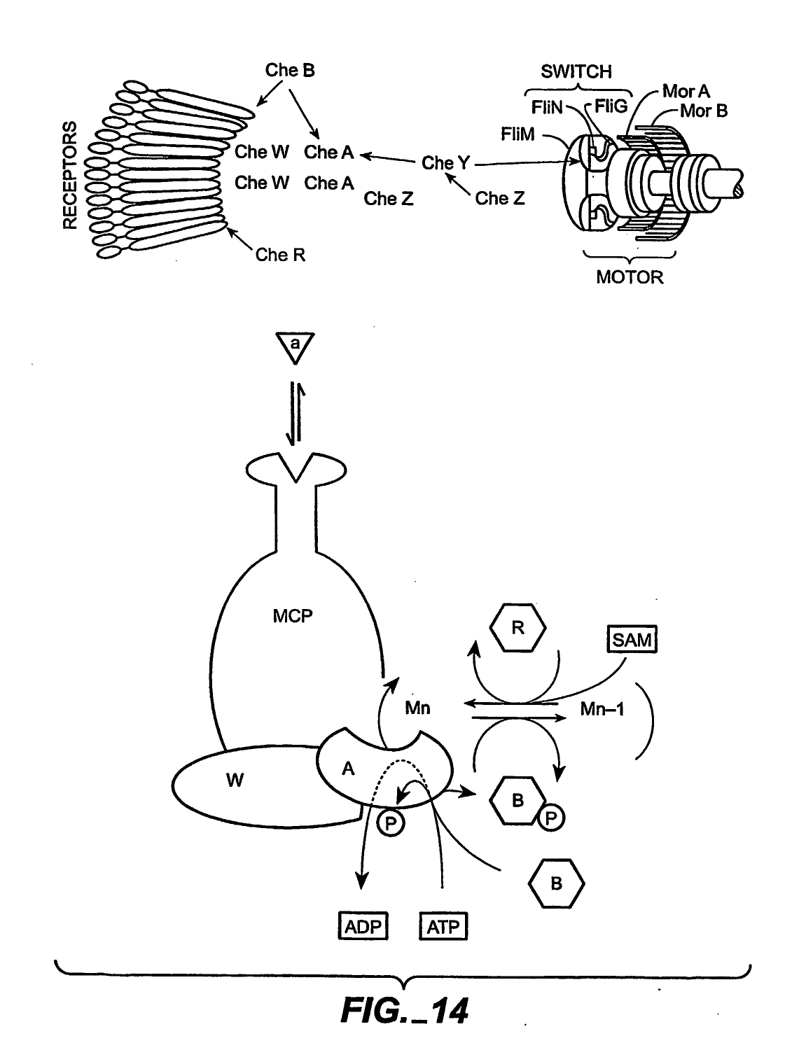

### Glossary

a and an (the indefinite articles) are used herein to mean “at least one” and “one or more,” and as used herein all of them: “a,” “an,” “at least one” and “one or more” have identically the same meaning.

activate is used herein to mean to increase or turn on, e.g., kinase activity.

adaptation, adaptation system means as used herein generally to adjust responsivity in accordance with ambient conditions, so that the signal generating system resets after a perturbation and maintains parity of signal generation with the parameter of interest by adjusting responsivity to reflect the current ambient level thereof. For instance, certain adaptive sensors of the preferred embodiments modulate the gain in the transduction of sensing moiety states (and/or changes therein) to signaling moieties reflective thereof, so that the signaling moieties engender the production of a detectable signal (and/or a change therein) only in response to a given increment of change in the ambient analyte concentration, which scales with the ambient level. In this way the system can be configured to produce a transient signal only when the concentration changes from the then current level (no matter what it may be) by more than a threshold amount. For example, a change in the ambient level of the parameter of interest impinges on the sensing moieties in a sensor and changes some of their states. The change in state of sensing moieties is transduced (individually or collectively) to the signaling moieties. The efficiency of transduction is determined by the modulation setting. When the increment of sensing state changes exceeds a threshold, it causes the signaling moiety to engender the production of a detectable signal indicative thereof, or it engenders a change in the signal indicative thereof. After a short time however, as a consequence of one or more of the events that engendered production of the signal, the modification that controls transduction is altered so that the new level of analyte no longer is sufficient to engender the detectable signal, which at this point is extinguished and is not produced again until the ambient analyte levels change again by at least the required threshold increment. At this point the system is said to have adapted to the changed environment. The threshold amount may be determined in absolute terms or relative to the initial or final levels over the immediate change interval. Thus, in a particular regard, adaptation is used herein to refer to the return of a system or organism to a preset state following a response to an altered environmental state or condition, even though the environment remains altered and does not itself revert back to the condition or state that existed before the alteration, and adaptation system is used herein to describe a mechanism that acts to modulate a function, such as to return it to a steady state value following a perturbation. An example is the system in chemosensoty systems in bacteria that restores the system to a preset state after a change in concentration of a stimulatoty ligand without an associated restoration of prestimulus ligand concentrations.

adaptation module see modification module, see adaptation, a module that comprises an adaptation moiety effective for modulating the transduction of a perturbation engendered by a change in state of a sensing moiety to a perturbation in a signaling moiety and in the production of a detectable signal thereby.

analog, variant, mutant, mutein is used herein to mean an altered version of a chemical compound (typically referred to as an analog or variant), such as a protein (typically referred to as a variant, mutein or mutant), a polynucleotide (often referred to as variants and mutants), cells (typically referred to as variants or mutants), organisms (typically variants and mutants) and systems.

analyte(s) is used herein to mean any measurable entity, chemical or physical, typically a physical variable or molecular entity.

associates, self-associates is used herein to mean binds, binds with self.

attractant as used herein refers to a chemical that causes a positive chemotaxis response.

bacteria as used herein refers to unicellular anuclear prokaryotic organisms of the Kingdom Bacteria, including Cyanobacteria, Gram Positive Bacteria, Proteobacteria (which includes gram negative bacteria, such as Salmonella sp. and Escherichia sp., including, in particular, for example S. typhimurium and E. coli), Thermatogales, and Spirochaetalae See, for instance, definition of bacteria, page 59, Oxford Dictionary of Biochemistry and Molecular Biology, Oxford University Press, Oxford, UK (1997).

To elaborate more extensively bacteria as used herein means the Bacteria, also referred to as the eubacteria, including the Actinobacteria, the Aquificae, the Bacteroidetes/Chlorobi group (including the Bacteroidetes and the Chlorobi), the ChlamydiaeNermcomicrobia group (including the Chiamydiae, the Verrucomicrobia and the Victivallis) the Chloroflexi (green non-sulfur bacteria), the Chrysiogenetes, the Cyanobacteria (blue-green algae) (including the Chroococcales, the Nostocales, the Oscillatoriales, the Pleurocapsales, the Prochlorophytes and the Stigonematales) the Deferribacteres, the Deinococcus-Thermus Group (including the Deinococci, the Dictyoglomi, the Fibrobacteres/Acidobacteria group (including the Acidobacteria and the Fibrobacteres), the Firmicutes (Gram-positive bacteria) (including the Bacilli, the Clostridia, and the Mollicutes), the Fusobacteria, the Gemmatimonadetes, the Nitrospirae, the Planctomycetes, the Proteobacteria (purple bacteria and relatives) (including the Alphaproteobacteria, the Betaproteobacteria, the delta/epsilon subdivisions, the Gammaproteobacteria), the Spirochaetes, the Thermodesulfobacteria, the Thermomicrobia, the Thermotogae (including the Thermotogae (class), the Caldithrix, the Cenibacterium, the Deinonema, the Denitromonas, the Ferribacter, the Guhaiyinggella, the KBI group, the Nanobacterium, the Natronohydrobacter, the Oceanicaulis, the Pseudogluconobacter, the RA21 methanotroph group, the SB1 group, the BRIO93 group, the Smegmaraptor, the Streptofusia and the Thermodesulfobiaceae). (Taken from the NCB1 website June 2003).

bind, binds, binding, bound, capture are used herein to mean the interaction and association together with one another of two or more distinct entities by a chemical, physical or physico-chemical bonding interaction whereby the entities are resident together at least some of the time over and above their mean free collisional association under the same conditions. The association can be reversible and weak, with the free and bound states of the entities in equilibrium with one another, the balance between the two shifting with the ambient environment and their chemical potentials in that environment (their molar concentrations to a first approximation). Where weak associations are involved in analyte binding, frequently an ensemble of receptors will be employed and the measure of the analyte will be the average fractional occupancy of all the available sites for binding the analyte of all the receptor molecules in the ensemble. At the opposite extreme as to strength of interaction and bonding together, the association may be strong and essentially irreversible, which may be the result of strong non-covalent interactions and/or it may involve covalent bonding. In general, except where binding (and the like) involves a process that results in addition of matter, such as bonding of a co-factor or conjugation of an adduct or addition of a covalent modification, or the release of matter, such as release of a part as a result of a cleavage reaction, the mass of the association formed by joining a number of distinct entities will be the sum of the masses of the individual entities prior to forming the association.

cassette as used herein means much the same thing as module.

CheA is used herein to designate the histidine kinases that carry out a central reaction in the processes that modulate motility in virtually all motile prokaryotes that have been examined. CheA is a cytoplasmic protein, and it typically carries out two distinct kinase reactions. First, it is autocatalytic, and its autocatalytic activity is responsive to ligand binding to MCPs. Second, CheA rapidly donates the phosphate added to a CheA histidine in the autocatalytic kinase reaction from CheA to CheY. CheY phosphorylation (at Asp57 in the E. coli system) generally reduces the affinity of CheY for CheA and increases its binding to components of the flagellar apparatus. Whatever the intermediate mechanisms, however, increases in CheY phosphorylation result generally in increased changes in direction of the organism.

CheW is used herein to refer to a typically cytoplasmic protein that generally binds to receptors and is required for receptor-dependent CheA kinase activation.

CheY is used herein to refer to a typically cytoplasmic protein that is phosphorylated by CheA and that generally in its phosphorylated state interacts with specific flagellar proteins to cause a change in flagellar activity that randomly causes motile cells to change their direction of travel.

CheZ is used herein to refer to a typically cytoplasmic protein that dephosphorylates phospho-CheY.

chemo-sensinca chemosensing is used herein to refer generally to processes by which substances are detected and/or measured, including the process by which cells detect attractants and repellents and processes by which sensing devices detect analytes including for instance, distinct chemical compounds, classes of compounds, compositions such as complexes containing two or more copies of a single compound, complexes made up of copies only of a given class of compounds, and complexes made of copies of multiple compounds.

chemotaxis is used herein generally to mean the tendency or proclivity of many cells to move toward some substances (attractants) and away from others (repellents), and the cell physiological and molecular processes and mechanisms involved in controlling cell motility and the direction of travel toward attractants and away from repellents.

chemotaxis protein is used herein to mean any protein component that is involved in chemotaxis, particularly those involved in the regulation of motility in response to attractants and repellents.

chimera is used herein generally to refer to a chimeric entity (see “chimeric”).

chimeric is used herein to mean made of elements that do not naturally occur with one another; a combination of things foreign to one another. The term “hybrid” is used herein in this sense to mean much the same thing as chimeric.

chimeric polypeptide is used herein to mean a polypeptide formed of at to least two polypeptides (or portions thereof) of foreign origin to one another. Chimeric polypeptides, such as fusion proteins, typically are linear chimeras in which the component polypeptides are arranged head to toe, single file. Chimeric polypeptides typically are not made by joining polypeptides per se although this can be done in some cases. Rather DNAs (or other polynucleotides in some cases) are made that express the polypeptides, by in frame fusion of polynucleotides that encode the desired amino acid sequences of the polypeptides and then express the resultant chimeric gene in a host cell or, occasionally, in vitro. Most often the chimeric gene is constructed in a suitable expression vector in vitro by standard recombinant DNA techniques, introduced into a desired cell and therein expressed, resulting in synthesis of the chimeric polypeptide. Genes to express chimeric polypeptides also can be made in other ways, however, including but not limited to in situ engineering of another gene in a host cell by in situ recombination methods. The gene can be an endogenous host cell gene or a gene that was previously transduced into the host. Both homologous and non-homologous in situ recombination methods have been developed and are widely known that can be used to engineer chimeric polypeptides. The term “hybrid polypeptide” is used herein in this sense to mean much the same thing.

chimeric sensor polypeptides is used herein to mean a chimeric polypeptide formed by the fusion of a polypeptide comprising a sensing moiety with another polypeptide, often a polypeptide comprising a signaling moiety; for example, a polypeptide formed by fusing a polypeptide from one MCP to a non-homologous polypeptide from a different MCP

cognate is used herein to means matched, matching, complementary, members of a matched set. For instance, a ligand and receptor that bind specifically to one another are a cognate pair. Any target analyte and analyte binding moiety that specifically bind to one another are a cognate pair.

complex is used herein to mean an assemblage of molecules and/or other entities, often as discussed herein primarily proteins, that function together, in some coordinated way, often as part of a system. The term also is used herein to mean an entity formed by the binding to one another of two or more component entities, such as: a ligand-receptor complex formed by association of a ligand and a receptor; an auxiliary protein-ligand receptor complex formed by association of a ligand with a cognate auxiliary ligand binding protein; a receptor signaling complex formed by the direct or indirect association with one another of a ligand, a cognate ligand-binding receptor protein, and a signaling protein; and a transmembrane signaling complex formed by the association of a periplasmic ligand with the periplasmic ligand-binding domain of a ligand-binding transmembrane receptor protein residing in and on both sides of a membrane and the association of the cytoplasmic domain of the receptor with a signaling protein.

comprising, comprised of, comprises and other words of the root “comprise” are used herein to mean that something is part of something else without implication or limitation as to anything else that may be included or left out. As used herein, therefore, “A is comprised of B” means that anything that is “A” includes something that is “B.” Furthermore, as used herein the phrase “A, comprising B” means exactly the same as “A is comprised of B.”

coupling is used herein to mean an act of connecting one entity, structure, activity, process or the like to another entity, structure, activity, process or the like, whereby one impinges on and/or affects the other. The connection may be direct or it may be indirect.

de novo is used herein to mean, generally, one of the following: essentially new; unrelated to what previously was known in a significant or material aspect, made from basic starting materials or essentially from basic starting materials (“from scratch”); not made or derived from a naturally occurring starting material of related or similar structure and/or function.

detectable signal is used herein to mean a measurable output (or its absence) or a measurable change therein, the output generally being a physical, chemical or physicochemical property.

Particularly useful as detectable signals in some of the preferred embodiments of the invention are products of a variety of enzyme catalyzed reactions that can be measured by absorption or emission of light, typically of defined wavelength or wavelength range. Examples in this regard include: the colored products produced by the action of enzymes on chromogenic substrates, such as those commonly used for ELISAs both in vitro and in situ—and absorption of light thereby, fluorescent products produced by the action of enzymes on fluorogenic substrates—and the fluorescence produced thereby; light produced by the action of luciferase and other luminogenic enzymes on luciferin and like substrates (respectively); and light produced by the action of enzymes on chemiluminogenic substrates.

Also particularly useful as detectable signals in some of the preferred embodiments of the invention are the products of a variety of non-enzymatic reactions that can be measured by absorption or emission of light, typically of defined wavelength or wavelength range. Examples in this regard include FRET-stimulated fluorescence produced by the association of a first entity comprising a donor fluorophore with a second entity comprising an acceptor fluorophore.

domain, domain in a polypeptide, domain in protein as used herein refers to any structurally and/or functionally distinct and/or definable region or regions of an entity, frequently in a polypeptide and/or protein, including, for instance: (a) sensing, receiving, analyte-binding, target-binding, attractant-binding, repellent-binding, N-terminal, periplasmic, N-terminal sensing, periplasmic sensing, ligand-binding, helix, alpha helix, four helix bundle, four helical bundle and inter helix region domains; (b) signaling, cytoplasmic, kinase, and histidine kinase domains; (c) transmembrane and intramembrane domains, and (d) linker and linking domains. A domain may be all or only a part of a structure. Thus, for instance, in dimer MCPs, the helix bundle-forming domains of each monomer is not, by itself, sufficient to form a four-helix bundle.

effector means as used herein that which engenders a response, such as cAMP and phosphorylated Che Y.

excitation means as used herein as to fluorescence and like processes of absorption and emission of electromagnetic radiation generally to refer to the elevation of electron(s) in a compound or electrons in a system from a first energy state to a second higher energy state, the second state typically being one from which the energy then is emitted in a characteristic manner, for instance, as light of a particular wavelength and/or polarization. As to systems, cells and organisms the term excitation is used herein generally to refer to the eliciting of a response by a stimulus, such as, for instance, a chemotaxis response of a motile cell stimulated by exposure to an attractant, repellent or a mixture thereof.

FRET, fluorescence resonance energy transfer, fluorescence energy transfer is used herein to mean the transfer of excited-state energy from an excited donor fluorophore to an acceptor fluorophore by long-range dipole-dipole interactions, without emission or absorption of a photon. The donor emission spectrum typically overlaps with the acceptor absorption spectrum. The donor and acceptor may be in the same molecule or in different molecules. The efficiency of FRET depends steeply on the distance between the donor and acceptor fluorophores, among other factors. FRET has found widespread use as a method for measuring distances in molecules and molecular complexes and as a detection reagent in a wide variety of assays, including hybridization assays, protein interaction assays, ligand binding assays and the like.

functionally homologous as used herein means homologous in function, homology of function. See also: “homolog,” “homology,” “homologous,” and “structurally homologous.”

heteromultimer, hetero-multimer generally denotes a multimer made up of different components, such as a heterodimer made up of two different monomers. See “multimer.”

higher order structure is used herein to mean spatial, physical and chemical features that characterize the arrangement and functioning of complex systems, such as large macromolecular assemblies.

homolog is used herein generally to refer to entities, usually polypeptides or proteins, so similar to one another in structure or function or both as to be considered, in at least that respect, related to one another and/or different versions of the same thing. See also: “functionally homologous,” “homologous,” “homology,” and “structurally homologous.”

homologous is used herein to mean so similar as to be considered related to one another, derived from a common origin (directly or indirectly), different versions of the same thing, and/or different versions of one another, as to the similarity. See also: “functionally homologous,” “homolog,” “homology,” and “structurally homologous.”

homology is used herein to mean similarity of structure or function or both sufficient for them to be considered derived directly or indirectly from a common origin, different versions of the same thing and/or different versions of one another as to the similarity. See also: “functionally homologous,” “homolog,” “homologous,” and “structurally homologous.”

homomultimer herein generally denotes a multimer comprised of a single type of component. Multimeric proteins comprised of polypeptides substantially identical to one another are examples of homomultimer. For instance, homomultimer is used herein to mean a protein composed of at least two identical subunits. (See “multimer.”)

hybrid, hybrid polypeptide the term hybrid is used herein in one sense to mean made up of two or more “parts” that originally were foreign to one another, such as a polypeptide formed by joining together in head to tail fashion so as to form a single, new, hybrid polypeptide, two otherwise disparate polypeptides (or portions thereof). The term is used in this sense to mean much the same thing as “chimeric.” (See “chimeric.”)

It should be noted, however, that the term has other meanings that are used herein as well as in other contexts. In particular, when a single stranded RNA pairs with a complementary single stranded DNA, the result is double-stranded RNA:DNA, which is referred to as a hybrid. The term has been extended to include the double stranded product formed by base pairing between any two complementary polynucleotides, and thus has come to include RNA duplexes, DNA duplexes, duplexes of PNAs and PNAs or RNAs or DNAs, and all other products that result from base pairing of complementary regions of different polynucleotide molecules when they are hybridized with one another.

identity is used herein to mean the same, identical.

indicative of, indicative, indicates; reflective of, reflective, reflects is/are used herein generally to mean that the subject provides reliable information about the object of the clause. In the statement “B that is indicative of A,” for instance, B is the subject, A is the object, and “indicative of” means that B provides reliable information about A. An example from the disclosure is “[a] signal indicative of the state of the sensing moiety . . . ” in which “signal” is the subject, “state of the sensing moiety” is the object and the phrase “indicative of” means that the signal provides reliable information about the state of the sensing moiety. The phrases necessarily therefore imply a causal relationship between the subject and the object so that “A reflects B” means that the existence and the state of A is indicated by the existence and the state of B.

information is used herein to mean any specification of the structural state of a system wherein distinctions are made between possible alternative structures, expressed in bits.

inhibit is used herein to mean to slow or retard, to prevent in part or in whole, or to block, such as to reduce or block an enzymatic activity or a chemical reaction.

intelligence is used herein to mean the ability to respond successfully to a new situation.

interaction is used herein to mean any transfer of state or information between two or more distinct structures or systems.

in vitro is used herein to mean outside the organism, ex vivo, in a test tube or other laboratory device, in cells in culture, in tissue culture.

lacl^{q} is used herein to mean the E. coli gene that encodes the q mutant of the lac repressor protein.

lacZ is used herein to mean the E. coli gene that encodes β-galactosidase.

lattice is used herein to mean a regular arrangement of units, generally molecular, in two or in three dimensions, such as a two-dimensional surface array, or the three-dimensional array of identical atomic or molecular units in a crystal.

learning is used herein generally to mean the acquisition of altered sensibilities (typically manifest as altered responses to a stimulus) from previous experience (typically of exposure to that stimulus or to other stimuli).

ligand is used herein to mean anything that binds to a cognate receptor, e.g., a small molecule such as an amino acid or sugar that binds to a protein at a specific binding site.

MCP is used herein as an acronym for “methylated chemotaxis protein” and “methyl-accepting chemotaxis protein.” See “methylated chemotaxis protein.”

memory is used herein generally to refer to an internal record of past experience.

methyl accepting chemotaxis protein, methylated chemotaxis protein, MCP(s) is used herein to mean the primary chemotaxis receptors in prokaryotes, usually transmembrane proteins, defined by a highly conserved C-terminal domain alpha helical structure that interacts with CheA and CheW.

methyl donor is used herein to mean a reagent that donates CH₃ groups, typically to a nucleophile, often a nitrogen or an oxygen atom. S-adensoylmethionine (“SAM”) is a cytoplasmic methyl donor found universally in biological systems.

methylation is used herein to refer to attachment of a methyl group to a substrate, and to the transfer of a methyl group from a methyl group donor to the substrate. Methylation of polypeptides, in many instances, including methylation of MCPs, involves the enzymatic transfer of a methyl group by a methyltransferase from a methyl donor (typically SAM) to a methyl-accepting substituent in one or more specific amino acids residues in the polypeptide chain.

methylation site is used herein to refer to a specific site of a methyl group attachment in a methyl accepting “substrate.” For instance, the methylation sites in MCPs are specific glutamate and glutamine residues in the highly conserved C-terminal domains of the receptors.

modification is used herein to mean a change, typically a structural alteration; often a structural modification in a sensor or sensor element, such as a structural alteration in a receptor that modulates transduction of an effect of the interaction of an analyte with a sensing moiety to impinge on a signaling moiety and stimulate it to engender the production of a detectable signal reliably indicative of the interaction. Typically in this regard the modification has at least two states and transduction is more efficient in one state than it is in the other. One example of a modification of this type is methylation in the conserved cytoplasmic domain of MCPs.

modulate, modulation is used herein to mean to vary, adjust, alter, change, or the like, such as, for instance, to increase the gain of a transduction event, so that, for a given input the output is either less or more; i.e., the strength of the output is less for the same input than it would be if it were modulated to increase the gain. As used herein for the most part, modulation refers to altering the coupling between the change in state of one or more sensing moieties and the resultant change in the detectable signal engendered by one or more signaling moieties reflective thereof. Adaptation is a particular variety of modulation.

modulation moiety is used herein to mean an entity for modulation, typically for modulating the effects of sensing moieties on signaling moieties. A moiety for sensor adaptation, for instance, is a modulation moiety that acts to vary the efficiency of transduction between sensing and signaling moieties so that the signaling moieties act to engender the production of a detectable signal only when there is a change in the state of the sensing moieties that exceeds a varying threshold defined by the current state of the moieties. See modulate, moiety, sensing moiety, signaling moiety.

modulation module is used herein to mean an entity (module, cassette or the like) that comprises a modulation moiety.

module see sensing module, modulation module, and/or adaptation module.

moiety, moieties is used herein to mean an entity characterized by a particular feature or features, which may be structural, functional or both structural and functional. Typically, a chemical structure, substituent, species, group or genus of substituents, having defined properties. See sensing moiety, signaling moiety, modulation moiety.

multimer is used herein generally to refer to a complex comprising two or more components of the same type or alike to one another in some way. Dimer, trimer, tetramer, pentamer, hexamer, etc. denote multimers containing, respectively, two, three, four, five, six, etc. of the components. Typical among multimers discussed herein are multimeric proteins comprised of two or more polypeptides. See “homomultimer” and “heteromultimer.”

non-naturally occurring is used herein to mean not occurring in nature, generally either differing in at least one aspect of structure from naturally occurring counterparts and/or differing in one or more ways in the circumstances of its occurrence from those in which it occurs in nature. The recitation is meant to exclude from the subject matter to which it refers all aspects of the subject matter that would be unpatentable a priori and aposteriori as being products of nature unaltered by the hand of man.

operably linked is used herein to mean joined so as to function properly in an intended fashion; linked so as to be operative, coupled as necessary to function.

peptide, polypeptide, protein, polypeptide and/or protein and related terms are used herein much in accordance with their art-recognized meaning as set out, for instance, in the Oxford Dictionary of Biochemistry and Molecular Biology, Oxford University Press, Oxford, UK (1997).

A number of aspects of their use herein should be noted, nonetheless.

First, it is to be noted that the terms as used herein include not only polypeptides made up of the predominating twenty naturally occurring amino acids but also polypeptides containing modified forms of these amino acids, unusual amino acids, modified or alternative backbone moieties and other variations on canonical polypeptide structure.

Second, it is to be noted that the term peptide as used herein denotes simply a short polypeptide. It specifically does not denote any structural distinction from polypeptides generally, and it does not imply any particular origin of peptides, such as synthesis or proteolysis.

Third, the terms polypeptide and protein are somewhat overlapping and are used together inclusively. For instance, polypeptides that make up monomeric proteins often are themselves properly denominated as proteins per se. Some proteins that might be called monomeric contain two or more polypeptides. Such proteins also might be called multimeric because they have more than one polypeptide in them. Many proteins are homomultimers; i.e., they are made up of two or more copies of the same polypeptide. Many other proteins are heteromultimers; i.e., they are made up of two or more different polypeptides.

Often, recombinant DNA expression is used to make monomeric or homomultimeric proteins. Expression of recombinant DNA typically results in the production, initially, of a single polypeptide. Unprocessed, the polypeptide may constitute a protein by itself, a true monomer protein. It will still constitute a monomeric protein if it is cleaved and only one of the fragments produced by cleavage makes up the resulting protein. As noted above, a protein made up of more than one fragment of a single proteolyzed polypeptide might be referred to as a homo/monomeric protein. It also might be referred to as a heteromultimeric protein, since the cleavage products that form the protein differ from one another, even though they were produced from a single original polypeptide. But “true” heteromultimeric proteins produced by recombinant DNA expression generally involve expression of more then one gene or the participation of a polypeptide produced by the host, in addition to that produced by expression of the recombinant DNA.

The terms polypeptide, protein, protein and or polypeptide and the like as used herein overlap and thus often mean the same thing as one another and include all of the above, unless it is explicitly indicated with respect to a given use that it means something else.

PhoA is used herein to mean an E. coli gene that encodes the enzyme alkaline phosphatase.

PhoB is used herein to mean an E. coli gene that encodes a transcription factor that is activated by aspartate phosphorylation and that (when activated) binds to upstream promoter regulatory sequences and controls expression of phoA

Prokarvote(s) also called the Procaryotae and the Monera, all as used herein mean all organisms in which genomic DNA is not enclosed in a nuclear membrane, and any organism whose cells possess a prokaryon. Prokaryotes include all Bacteria and Archaea, inter alia. See the definition of “prokaryote” on page 534 of the Oxford Dictionary of Biochemistry and Molecular Biology, Oxford University Press, Oxford, UK (1997). Also see the definition above of the term “bacteria.”

protein-hand complex as used herein means a complex comprised of a ligand bound to a protein, which may also comprise auxiliary proteins and/or other components that bind to the ligand, the protein, or both.

receptor as used herein generally means an entity that specifically interacts with a particular aspect of the environment and is involved in generating a signal indicative of its interaction therewith. Receptors discussed herein often are membrane-spanning proteins that bind specific ligands and transmit (directly or indirectly) a signal into the cell indicative of the absence, presence or amount of the ligand in the environment. A particular type of receptor discussed herein is the MCP (methylated chemotaxis receptor). MCPs span the cell membranes of motile prokaryotes, interact with specific ligands on the exterior side of the membrane and, on the cytoplasmic side, influence the activity of a kinase that determines, through a number of intermediate events, the tumbling activity and, thus, the general direction of travel of the cell.

receptor protein. receptor polypeptide, receptor polypeptide and/or protein as used herein means a protein, polypeptide, and/or polypeptide and/or protein that functions as a receptor. See receptor.

reflective of, reflective, reflects See “indicative of, indicative, indicates.”

repellent is used herein generally to refer to a chemical that causes a negative chemotaxis response.

sensing as used herein means the acquisition of information, in particular in regard to certain aspects and preferred embodiments of the invention, the term means the acquisition of information about the presence and/or the amount of a target analyte in an ambient environment by a specific, selective, preferential and/or exclusive interaction of a sensing moiety with the target analyte and a change in the state of the sensing moiety—or a change in the average state of a plurality of sensing moieties—in response to a change in the presence and/or the amount and/or the chemical potential of the target analyte in the ambient environment.

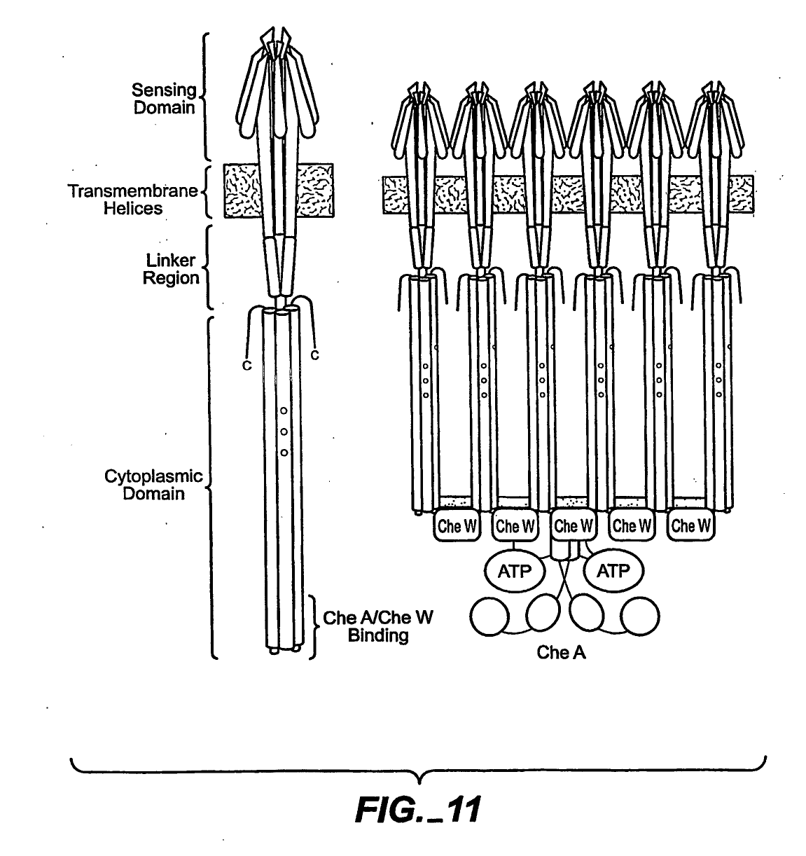

sensing domain means as used herein a domain, typically of a polypeptide and/or protein that forms, by itself or in association with other domains, a sensing moiety and/or a sensing module. The distinction between the three terms is illustrated by the generalized structure of a transmembrane MCP. The MCP depicted in FIG. 11, for instance is a homodimer. Sensing domains in the two identical polypeptides in the homodimer associate with one another to form a four helix bundle that comprises a sensing moiety. The sensing moiety is comprised in a sensing module, also formed substantially by the four-helix bundle forming domains of each of the associated polypeptides.

sensing module as used herein means an entity comprising a sensing moiety. Sensing moieties may be comprised in sensing modules to facilitate ease of use, utility, function, manufacture, modification or other features of the sensing moiety and its use. The term sensing module is used herein frequently to mean sensing moiety-comprising polypeptide and/or protein structures that facilitate the interchange, transfer, alteration, modification, use, joining and fabrication of signaling and sensing moieties, as well as other moieties such as modulating moieties.

By way of illustration, a sensing module serves as a container or cassette that accommodates both the structural and functional requirements of the sensing moiety and that also provides structural and functional features that facilitate use. For instance, a sensing module derived from or based on an MCP generally would comprise the MCP sensing moiety (typically made up of the sensing domains in the MCP, which form the functional sensing moiety), together with a substructure for fusing the sensing module to other structures for making a functional receptor and associated structures. Typically, the structures in such polypeptide and/or protein modules that facilitate fusing them to other structures are engineered into a DNA as linker regions and splice sites for joining disparate DNAs together to form genes that encode desired fusions having, as well, the structures required in the polypeptide fusion for it to function properly.

sensing moiety as used herein means an entity that interacts with one or more analytes. Generally a sensing moiety interacts preferentially, selectively, specifically, substantially exclusively or exclusively with one specific analyte, a limited number of specific analytes or a specific group of analytes. A sensing moiety and the specific analyte or analytes with which it interacts are referred to as: cognates of one another, cognate sensing moiety and cognate analyte, cognate pair, etc.

In addition, in general, the sensing moiety is in one state when interaction with cognate analyte is not taking place or is below a certain threshold level, and it is in a different state when interaction with cognate analyte meets or exceeds a certain threshold. Change in conditions that decrease interaction from above to below the threshold level or that increase interaction from below to above the threshold cause the state of the sensing moiety to change. In typical sensors the state of the sensing moiety, or a change in its state, is transduced and thereby impinges on one or more signaling moieties (either alone or in combination with the transduction of states of other sensing moieties). As a result, the detectable signal engendered by the signaling moiety is influenced by and reflects the interaction of one or more sensing moieties with their cognate analytes.

A sensing moiety generally interacts with its cognate analyte or analytes through chemical interactions, predominantly; but, it may do so through physicochemical or physical interactions or a combination thereof. Generally, the interaction between sensing moieties analytes involves non-covalent chemical interactions, but, it may do also involve covalent bonding as well.

The interaction between a sensing moiety and cognate analyte may involve formation of a short lived transient analyte-sensing moiety complex, a longer-lived transient complex, a stable complex, an essentially non-dissociating non-covalent complex or a covalently bonded complex, to name a few. The forward and back rates of the interactions vary greatly for different cognate sensing moieties and analytes.

In a given sensor in accordance with the invention, many sensing moieties are available to interact with cognate analytes, and production of a detectable signal reflects statistical parameters of the interactions of the plurality of available sensing moieties with cognate analytes in the “environment” to which the sensor is exposed.

Different sensor designs make use of different binding properties and kinetic characteristics of cognate pairs of sensing moieties and analytes and, moreover, use different combinations of sensing moieties to detect and/or measure one or more cognate analytes over narrow and/or broad ranges of occurrence, alone or in combination, reversible or irreversibly. As a result, the invention provides sensors that can match just about any type of analyte-sensing requirement.

The analytes can be chemical in nature as described elsewhere herein, such as ligands (including, for instance, chemosensory attractants, repellents) and ligand-binding auxiliary proteins, peptides, polypeptides, proteins, polynucleotides, cell surface markers, proteoglycans, carbohydrates, lipid adducts, or a wide variety of other compounds, substances, complexes, polymers and the like. The analytes also can be physical phenomena and/or properties, such as temperature, pressure, light, and motion, to name just a very few.

sensor(s) as used herein means, generally, a composition, apparatus, device or the like for detecting and/or measuring a substance or substances, or a physical entity such as light or other electromagnetic radiation The term as used herein is inclusive of various other names for sensors and of particular types of sensors, including chemosensors and biosensors. It is used herein frequently in reference to novel sensors in accordance with various aspects and preferred embodiments of the invention herein disclosed.

sensor element as used herein means a structure (real or virtual) comprising at least one of a sensing moiety, a signaling moiety and/or modulating moiety. In preferred aspects of the invention in this regard, a sensor moiety comprises a sensing moiety and a signaling moiety. In certain other preferred embodiments of the invention sensor elements comprise all three moieties.

signaling domain means as used herein generally to denote a continuous or discontinuous region of a polypeptide or other molecule or multimeric protein or other multimeric complex or composition that produces or engenders or controls the production of a detectable signal.

signaling moiety as used herein means an entity that engenders, produces, results in, modulates, or otherwise initiates or controls the production of a detectable signal. An example of the activity of a signaling moiety is the active site of a dimeric kinase, such as CheA, comprised of the kinase domains of each of the monomers.

state of the sensing moiety and changes thereto means as used herein the condition of a physical system with regard to phase, form, composition or structure such as those qualitatively or quantitatively indicative of analyte binding thereto.

structurally homologous is used herein to mean homologous in structural or structural features, homology of structure. See also: “functionally homologous,” “homolog,” “homology,” and “homologous.”

substantially as used herein means in large part, for the most part, almost all, just about, all but an insubstantial portion, most, more than most, just about all, virtually all, as to substance, all substantive aspects, importance, value, degree, or extent; short of everything, but, missing nothing important.

target, target analyte as used herein means the objective, the desired object, that which is being sought, the intended goal. An example of the use of the term herein is “target analyte” by which is meant the analyte that is intended to be detected and/or determined and/or measured in amount, concentration, chemical potential or the like. A sensing moiety specific for a target analyte and the target analyte are a cognate pair, and typically one or more cognate sensing moieties would be able to detect a given target analyte.

transduce, transduced, transduction as used herein refers to the coupling of effects so that an event at a first locus causes an effect at a second locus. For instance, in sensors of the present invention, a change in state of a sensing moiety at a first locus, generally, for instance on the outside of a cell membrane, causes a change in the action of a signaling moiety at a second locus. Since the state or change in states causes a corresponding change in the action of the signaling moiety, under certain defined circumstance, the two are coupled and, without knowing the mechanism, the state change of the sensing moiety is transduced to impinge on the action of the signaling moiety in example. The mechanism of transduction need not be known, and in most cases has not be elucidated. It clearly generally is not mediated by direct contact between the sensing moiety and the signaling moiety. Nor, in most cases, is it mediated by direct action of the former on the latter. Rather, coupling between sensing moiety state and the activity of the signaling moiety very likely involves a chain of events and a number of intermediaries that is only initiated by the state or state change of the sensing moiety.

transmembrane receptor polypeptide and/or protein as used herein means a polypeptide and/or protein that ordinarily spans a membrane with one part residing in the membrane, one part exposed on one side of the membrane and another part exposed on the other side of the membrane. MCPs provide examples of transmembrane proteins. E. coli MCPs are disposed on both sides of the cell membrane. The ligand binding domain lies outside the membrane (at least in part) exposed to the periplasm so that it can bind cognate ligand entering from the surrounding environment. The signaling domain lies on the other side of the membrane (at least in part), where it contacts auxiliary proteins involved in modulating the flagella switch in response to ambient ligands. The remainder of the protein resides largely or entirely within the membrane.

In MCPs and other, like, polypeptides and proteins that can be used in the invention herein disclosed in the same way, the ligand binding domain comprises a sensing moiety that interacts with a cognate ligand. In certain preferred embodiments of various aspects of the invention, when the cognate ligand is present, or is present at or above a certain minimum threshold, or changes by more than a threshold increment from the then current level, the interaction of the ligand and the sensing moiety engenders a change in the “state” of the sensing moiety, and the state change is transduced from the sensing moiety to the signaling moiety on the other side of the membrane, which engenders the production of a detectable signal. The exact mechanism of transduction is not known but appears to involve transmembrane signaling.

triggering means as used herein starting or initiating, such as the production of a response that occurs when the concentration of a molecule or the value of a physical parameter reaches a critical value.

### DETAILED DESCRIPTION AND PREFERRED EMBODIMENTS

It is among the objects of the invention to provide in certain of its preferred aspects and embodiments thereof, improved sensors, and compounds, compositions, methods, devices, systems, uses and the like relating thereto.

In a particular aspect in this regard, certain preferred embodiments of the invention provide a sensor comprising a sensing moiety that interacts and/or binds with one or more target analytes and a signaling moiety that engenders the production of one or more detectable signals.

And/or wherein interaction of target analyte with the sensing moiety determinably alters the production of one or more of the detectable signals engendered by the signaling moiety.

And/or wherein the sensing moiety and the signaling moiety are heterologous to one another and/or the sensing moiety and the signaling moiety do not occur naturally with one another in the same cell and/or the sensing moiety.

And/or wherein the sensing moiety and/or the signaling moiety is not a naturally occurring entity.

And/or wherein the sensing moiety is comprised in a receptor with a domain heterologous thereto.

And/or wherein the signaling moiety is comprised in signaling entity with a domain heterologous thereto.

And/or wherein the sensing moiety, the signaling moiety and/or both the sensing moiety and the signaling moiety are not naturally occurring moieties.

And/or wherein the sensing moiety is a de novo sensing moiety.

And/or wherein the sensing moiety is a de novo four helix bundle binding domain of a receptor polypeptide and/or protein or a fragment, derivative, variant or modified form thereof.

And/or wherein the alteration is an increase in intensity, a decrease in intensity, a change in frequency or a change in periodicity of the detectable signal engendered by the signaling moiety.

And/or wherein the alteration is a change in polarization, a change in luminescence, a change in phosphorescence, a change in fluorescence, a change in absorption, a change in electron spin resonance, a change in magnetic resonance.

And/or wherein interaction of at least one target analyte with at least one sensing moiety determinably induces the production of at least one detectable signal engendered by the signaling moiety.

And/or wherein at least one sensing moiety and at least one signaling moiety are comprised in the same molecule.

And/or wherein at least one signaling moiety and at least one sensing moiety are comprised in different molecules.

And/or wherein at least one sensing moiety is comprised of one or more signaling domains.

And/or wherein at least one sensing moiety is a multimer comprised of n sensing domains disposed in m molecules, where n and m are the average stoichiometries and at least one or n and m is greater than 1.

And/or wherein at least one sensing moiety is a multimer comprised of n sensing domains disposed in m molecules, where m and n are integers greater than 1 and the ratio of m to n is 1 or 2.

And/or wherein at least one sensing moiety is comprised of a homomultimer.

And/or wherein at least one sensing moiety is comprised of a heteromultimer.

And/or wherein at least one sensing moiety is comprised of a homodimer, heterodimer, a homotrimer, a heterotrimer, a homotetramer, or a heterotetramer.

And/or wherein at least one sensing moiety is comprised of a first plurality of n sensing domains comprised in a second plurality of m molecules, wherein n is an integer above 1, m is an integer above 1, and the ratio of n to m is a non-zero positive integer.

And/or wherein the sensing moiety is comprised of a homodimer, a heterodimer, a homotetramer, or a heterotetramer of molecules each of the molecules comprising a sensing domain.

And/or wherein the sensing moiety is comprised of one or more sensing domains and the sensing domains are comprised in one or more polypeptides.

And/or wherein the sensing moiety is comprised of one or more sensing domains and the sensing domains are comprised in one or more chimeric polypeptides.

And/or wherein the sensing moiety is comprised of one or more sensing domains and the sensing domains are comprised in one or more chimeric polypeptides.

And/or wherein the sensing moiety is comprised of one or more sensing domains and the sensing domains are comprised in one or more chimeric polypeptides, wherein one or more sensing domains in one or more chimeric polypeptides is comprised of a region of a sensing domain synthesized de novo.

And/or wherein one or more sensing domains are those of one or more sensing domains synthesized de novo.

And/or wherein one or more of the sensing domains is that of a synthesized polypeptide comprising a region having a de novo designed binary patterned sequence.

And/or wherein the sensing moiety is comprised of one or more sensing domains and the sensing domains are comprised in one or more chimeric polypeptides, wherein one or more sensing domains in one or more chimeric polypeptides is comprised of a region of a sensing domain of a polypeptide obtained through a process comprising one or more steps of in vitro evolution.

And/or wherein one or more sensing domains are those of one or more sensing domains of a four helix bundle protein.

And/or wherein the sensing moiety is comprised in a multimer comprising two or more chimeric proteins, each of the two or more chimeric proteins comprises one or more sensing domains, and at least one of the sensing domains in each of the two or more chimeric proteins comprises one or more of the helix forming domains of one or more polypeptides that form a four helix bundle.

And/or wherein the sensing moiety is comprised in a multimer comprising two or more chimeric proteins, each of the two or more chimeric proteins comprises one or more sensing domains, and at least one of the sensing domains in each of the two or more chimeric proteins comprises one or more of the helix forming domains of one or more polypeptides that form a four helix bundle protein of a four helix bundle-forming receptor protein.

And/or wherein the sensing moiety is comprised in a multimer comprising two or more chimeric proteins, each of the two or more chimeric proteins comprises one or more sensing domains, and at least one of the sensing domains in each of the two or more chimeric proteins comprises one or more of the helix forming domains of one or more polypeptides that form a four helix bundle protein of a four helix bundle-forming receptor of a chemotaxis receptor protein.

And/or wherein the sensing moiety is comprised in a multimer comprising two or more chimeric proteins, each of the two or more chimeric proteins comprises one or more sensing domains, and at least one of the sensing domains in each of the two or more chimeric proteins comprises one or more of the helix forming domains of one or more polypeptides that form a four helix bundle protein of a four helix bundle-forming receptor of a feedback modulated receptor protein.

And/or wherein the sensing moiety is comprised in a multimer comprising two or more chimeric proteins, each of the two or more chimeric proteins comprises one or more sensing domains, and at least one of the sensing domains in each of the two or more chimeric proteins comprises one or more of the helix forming domains of one or more polypeptides that form a four helix bundle protein of a four helix bundle-forming receptor of a methylated receptor protein, wherein methylation of one or more of the sensing domains alters the relationship between target analyte interaction with the sensing moiety and the determinable change in the production of a detectable signal engendered by the signaling moiety.

And/or wherein the target analyte binds to the sensing moiety.

And/or wherein the sensing moiety binds specifically to the target analyte with high affinity.

And/or wherein the complex of sensing moiety bound to the target analyte has a dissociation constant above 10⁻⁹, 10⁻¹⁰, 10⁻¹¹, 10⁻¹², 10⁻¹³ or 10⁻¹⁴.

And/or wherein the sensing moiety binds a plurality of analytes.

And/or a sensor according to any of the claims herein^{I}, wherein each complex formed by binding of the sensing moiety to a target analyte of the plurality of target analytes has a dissociation constant below 10⁻⁹, 10⁻¹⁰, 10⁻¹¹, 10⁻¹², 10⁻¹³ or 10⁻¹⁴.

And/or comprising a first plurality of sensing moieties each of which binds a second plurality of target analytes.

And/or comprising a first plurality of sensing moieties each of which binds a second plurality of target analytes with high specificity.

And/or comprising a first plurality of sensing moieties each of which binds a second plurality of target analytes, wherein the dissociation constant of the complexes formed by binding of sensing moieties of the first plurality to target analytes of the second plurality is above 10⁻⁹, 10⁻¹⁰, 10⁻¹¹, 10⁻¹², 10⁻¹³ or 10⁻¹⁴.

And/or comprising a first plurality of sensing moieties each of which binds a second plurality of target analytes, wherein the dissociation constant of the complexes formed by binding of sensing moieties of the first plurality to target analytes of the second plurality is above 10⁻⁹, 10⁻¹¹, 10⁻¹², 10⁻¹², 10⁻¹³ or 10¹⁴.

And/or wherein a threshold plurality of target analytes interacting with a corresponding plurality of sensing moieties is required to produce a determinable alteration in the production of a detectable signal engendered by one or more signaling moieties.

A method for detecting a target analyte comprising contacting a composition of matter and/or sample in which the target analyte is to be detected with a sensor comprising a sensing moiety and a signaling moiety, wherein the sensing moiety interacts with target analyte in the sample, the signaling moiety engenders the production of a detectable signal, the interaction of the target analyte with the sensing moiety determinably alters the production of a detectable signal engendered by the signaling moiety, and the resulting detectable signal is thereby indicative of the presence or absence of a threshold amount and or concentration of target analyte in the sample.

And/or wherein the target analyte binds to the sensing moiety.

And/or wherein the binding of target analyte to the sensing moiety induces the production of a detectable signal engendered by the signaling moiety.

And/or any of the foregoing and/or wherein optionally still further, to provide improved signal to noise ratio of the detectable signal, the combination value at any time, tᵢ, reflects the average value over an interval Δtᵢ, where Δtᵢ, is small compared to tᵢ and large compared to transient fluctuations in the states of the sensing moieties.

The invention disclosed herein relates generally to, among other things, novel sensors and to methods, compositions, materials, components, devices, and systems relating thereto. It includes novel sensors. It also includes, to name but a few features, novel moieties, modules, compositions, materials, components, assemblies, devices, methods and systems for making and using sensors, including, in particular, the novel sensors. And it further includes, among others, methods for making and using the foregoing novel moieties, modules, compositions, materials, components, assemblies, devices, methods and systems. These and other aspects, features and embodiments of the invention are described herein both in general terms, by generic exemplification and by specific examples. However, the disclosure is not encyclopedic of the invention's features and embodiments and a full understanding of the invention can be gained from the disclosure only when it is read and studied in its entirety with the knowledge and understanding of the skilled practitioner in the arts to which it pertains. By way only of illustrative disclosure, therefore, in some of its many aspects, facets, features and embodiments, the invention provides as follows.

Sensors

Sensors of the invention typically comprise a sensing moiety that interacts with a target analyte and a signaling moiety that engenders the production of a detectable signal. Interaction of target analyte with the sensing moiety affects the signaling moiety and the detectable signal and/or changes therein reflect the “ambient” level of target analyte exposed to the sensing moiety.

Sensor Elements and Elemental Sensors

A sensor element is an entity (real or virtual) that is or that comprises at least one of the following: a sensing moiety, a signaling moiety and/or a modulating moiety. In preferred embodiments of the invention in a variety of aspects in this regard, a sensor element comprise a sensing moiety and a signaling moiety. In certain of the preferred embodiments of related aspects of the invention, sensor elements comprise a sensing moiety, a signaling moiety and a modulating moiety. In certain of the preferred embodiments of various further related aspects of the invention sensor elements comprises other moieties alone, in combination with one another and/or in combination with one or more of a sensing, signaling or modulating moiety. In preferred embodiments in some aspects of the invention sensor elements function as sensors independently of other sensor elements. In other preferred embodiments in related aspects of the invention, sensor elements cannot by themselves function as sensors and instead function as sensors together with other sensor elements in a complex, matrix or higher order structure. In further preferred embodiments in related aspects of the invention, sensors are comprised of both sensor elements that function as elemental sensors independently of one another, and of sensor elements that function as elemental sensors only in combination with one or more other sensor elements.

In certain further preferred embodiments the sensing moiety directly and/or indirectly affects the activity of the signaling moiety that directly or indirectly results in the production of a detectable signal and the detectable signal qualitatively and/or quantitatively is indicative of the absence or presence of the amount of the analyte.

The invention provides sensors as described herein for, in particular, (i) detecting, qualitatively, whether a target analyte is absent or present and/or, (ii) determining, qualitatively and/or quantitatively, whether a target analyte is present in an amount, concentration, density or the like equal to or exceeding, or equal to or less than a threshold value, and/or (iii) determining, quantitatively, the amount, concentration, or the like, if any can be detected, of the target analyte.

In preferred embodiments of the invention in another aspect, the sensing moiety is heterologous in origin and/or sequence to the signaling moiety and/or the sensing moiety and/or the signaling moiety or both do not occur in nature (i.e., is/are non-naturally occurring). These and other illustrative examples of various preferred embodiments of various aspects of the invention are discussed in greater detail below.

Interchangeability and Multiplicities of Moieties

In certain preferred embodiments of the invention the sensors generally are comprised of a first module comprising a sensing moiety that interacts directly and/or indirectly with an analyte to be determined and a second module comprising a signaling moiety that directly and/or indirectly produces or engenders the production of a detectable signal.

In preferred embodiments of one aspect of the invention, the sensing and signaling moieties are modular, a variety of sensing moieties specific for different analytes can be used with the same signaling moieties, and a single underlying signaling system can be adapted by switching sensing moieties to detecting and quantifying virtually an unlimited variety of analytes. In much the same way, by this aspect of the invention, a variety of signaling moieties can be used with a single sensing moiety. The invention thus provides significantly improved methods and capabilities to implement new sensor specificities by introducing new sensing moieties conveniently and without, necessarily, altering other structures.

In yet still further preferred embodiments in this regard, different sensing moieties (e.g., for detecting different analytes) can be used virtually interchangeably with a given signaling moiety, without substantially altering either moiety and without substantially deleteriously altering the direct and/or indirect “coupling” of the two moieties through which interaction of the sensing moiety with analyte affects the direct and/or indirect production of a detectable signal by the signaling moiety.

In especially highly preferred embodiments in this regard, a variety of first and second modules can be used together interchangeably without substantially altering either module and without adversely affecting the coupling between signal generation and the interaction of the sensing moiety with target analyte. Further in this regard, in certain of the highly preferred embodiments, the same given signaling moiety can be used with a great many sensing moieties. Likewise in accordance with certain highly preferred embodiments, the same given sensing moiety can be used with many different signaling moieties.

In another aspect of the invention, preferred embodiments provide sensors comprising a plurality of different sensing moieties and one or more signaling moieties, wherein the sensing moieties are specific for the same target analyte. In a related aspect of the invention, preferred embodiments provide sensors comprising a first multiplicity of sensing moieties for specifically detecting a second multiplicity of target analytes, wherein there is one or more different sensing moieties specific to each target analyte and each of the different sensing moieties is present in one or more instances. The invention in this regard makes possible the use in combination of a variety and number of sensing and signaling moieties, and it provides sensors that can, for instance, utilize several different sensing moieties specific to a specific target analyte or use several different sensing moieties specific to several different target analytes. The former can provide more robust detection of a specific target analyte under varying conditions and more selectivity over spuriously cross reacting material, for example. The latter provides multiplex sensors that detect and quantify several analytes at once.

Modulation

In certain very highly preferred embodiments in accordance with the foregoing, the interactions of the sensor elements in a given complex further are variable. In accordance with this aspect of the invention, the binding of a target analyte to a given sensing moiety in a complex can have a range of effects on the activity of the signaling moieties in the complex in engendering the production of a detectable signal. Moreover, in certain preferred aspects and embodiments of the invention in this regard, the “coupling” between target analyte binding to a given sensor element and its effect on the activity of the signaling moieties is varied by more than one mechanism.

In preferred aspects of the invention in this regard the sensors comprise a modulation moiety, preferably modular and particularly preferably comprised in a modulation module.

In a related aspect of the invention in certain other preferred embodiments, the “coupling” (or gain) between binding of a target analyte to a given sensing moiety and the effect thereof on the signaling moieties in engendering the production of a detectable signal can be modulated in response to the environment. In particular in this regard preferred embodiments can be modulated in response to the ambient level of one or more target analytes. In certain of the preferred embodiments in this regard the “coupling” is modulated for one or more sensing moieties in one or more complexes in the sensor so that the change in the level of analyte concentration required to generate a change in the production of a detectable signal is a constant fraction of the ambient concentration of the target analyte. This can be the case for one, for some, or for all of the sensing moieties in one or more or all of the complexes in a sensor in accordance with this aspect.

Adaptation

In another aspect of the invention, preferred embodiments provide adaptive sensors, particularly sensors in which the production of a detectable signal engendered by the signaling moiety is indicative of change in the level of target analyte relative to the ambient level. In particularly preferred embodiments in this regard, the invention provides sensors in which the production of a detectable signal, or a determinable alteration therein, is indicative of a threshold level of the target analyte and, in adaptive sensors of the invention, the threshold level varies so that it is always a set increment different from the contemporaneous ambient level. The set increment in certain highly preferred embodiments in this regard is a fixed amount; but, in some highly preferred embodiments, it varies with the ambient level of the target and preferably is a relative increment of the ambient level of the target. In some preferred embodiments in this regard, the relative increment is fixed, such as a fixed percent. In other preferred embodiments in this regard, the relative increment varies. Adaptation provides novel sensors of the invention in which, in certain especially preferred embodiments in this regard, production of the detectable signal or determinable alterations therein is indicative of the first or second order differential of the time series of the target level in the time period immediately incident to the time the alteration occurs. In other especially preferred embodiments, production of the detectable signal or the determinable alteration therein varies with the log of the change in the ambient level of the target analyte.

Adaptational Modulation and Modules

The adaptation function is supplied by a third module of protein structure. This module can be modified to control the relationship between the analyte sensory cassette function and the output of a given set of associated signaling cassettes. The adaptation module can be varied at will to control the nature of the sensory-response relationship so that this structure provides a third cassette that may be interchanged at will to provide different modalities of sensor function, including among others, changes in threshold sensitivity, changes in the effective algorithms for signal output for multiplexed sensors, and finally, with associated regulatory elements, the possibility for various continuously variable stimulus-response connectivities that allow previous environmental inputs to control the outputs produced in response to real time signals. The design and production of numerous variants of these three cassettes, sensing, adapting, and signaling, allows the production of nanoscale signal relay devices with an unprecedented range of gating characteristics that can be used to design and construct nanoscale computational devices. The tunable stochastic aspects of these gates make them particularly suited to the design of so called quantum computational devices.

Multiplicity, Variety, Numerousity, Complexes. Matrices, Higher Order Structures

Certain particularly preferred sensors of the invention are comprised of complexes. Each complex is comprised of, generally speaking, a plurality of sensor elements. The sensor elements comprise one or more sensing moieties and one or more signaling moieties. The sensing moieties in a given sensor element may be specific for one or for more than one target analyte. The signaling moieties in a given complex may be the same or different and may engender the production of one or more than one detectable signal. Moreover, each sensing moiety in a given complex may or may not be directly coupled to a signaling moiety. Likewise, each signaling moiety in a given complex may or may not be directly coupled to a sensing moiety. In accordance with certain preferred embodiments of the invention, the sensor elements in each complex interact directly or indirectly. Binding of a given target analyte to a given sensing moiety alters these interactions. The interaction of the sensor elements in a given complex affects the activity of the signaling moieties of the complex in engendering the production of detectable signals. The binding of target analyte to sensing moieties in a given complex, thus, controls the production of the detectable signal.

For instance, in certain other preferred embodiments relating to methyl-modulated chemosensory receptors, multiple sensing moieties interact with a given MCP to produce responses to multiple distinct classes of analyte ligands. These moieties can be contained in auxiliary primary sensing protein modules such as a periplasmic binding protein, as well as in a sensory moiety contained in the MCP. In these types of embodiments a single sensor element can produce detectable signals in response to two or more distinctly different analytes separately or in combination. A particular example of this embodiment is provided by the E. coli Tar MCP whose sensory moiety binds aspartate to effect a detectable stimulus, and additionally binds the maltose bound form of the periplasmic binding protein for maltose to produce a detectable signal. In response to both aspartate and maltose this sensor element produces a signal that is an integrated effect of both ligands acting together. An illustrative example in this regard and techniques useful for making and using such multiple analyte-capable sensor systems is provided by Mowbray and Koshland (1987), Cell. 50:171-180, in their report on “Additive and independent responses in a single receptor: aspartate and maltose stimuli on the tar protein,” which is incorporated herein by reference in its entirety particularly in parts pertinent to hybrid sensors and multiple analyte detection as discussed immediately above and elsewhere herein.

Indirect Analyte Binding

A wide range of periplasmic ligand binding proteins occur in prokaryotes. These proteins primarily function as the first stage of transport of nutrients into the cell. A subset of the binding proteins also interact specifically with chemotaxis receptors to promote chemotaxis towards the ligand of the binding protein (Quiocho and Ledvina, 1996). In E. coli for example, the Trg and Tap chemosensory receptors do not bind their cognate ligands directly. Rather, the ligands first bind to cognate periplasmic binding proteins and the complex of ligand and periplasmic protein then binds specifically to its cognate chemoreceptor. In the absence of bound ligand, the periplasmic protein does not bind to the receptor, as is the case for E. coli Trg and Tap. See Falke et al. (1997) which is herein incorporated by reference in its entirety, particularly in parts pertinent to auxiliary proteins of bacterial chemosensory systems and their use in accordance with various aspects and preferred embodiments of the present invention. Examples of such proteins include ribose binding and maltose binding proteins.

It is of interest to various aspects of the present invention that auxiliary protein from different cells that bind the same ligand do not necessary interact functionally with the other cells cognate chemoreceptor. For instance, in both E. coli and Salmonella maltose binds to an auxiliary protein first and the resulting complex, not maltose itself, binds to the Tar receptor. The mechanism is much the same in the two different types of cells in this regard; but, the maltose binding proteins from both cells interact with E. coli Tar but not with Salmonella Tar (Mizuno et al., 1986).

The interaction between the periplasmic binding domain and any binding protein of interest can be evolved from proteins with the same geometry as Tar, and it may be possible in a variety of circumstances to evolve specific protein-protein interactions more readily than small molecule-receptor interactions. The use of auxiliary binding proteins, thus, can provide significant advantages over direct binding approaches in such circumstances.

In certain preferred embodiments of the invention the sensing moiety is contained within an auxiliary protein that is distinct from, but interacts with, the N-terminal region of an MCP so that this portion of the MCP functions as a sensing module for the analyte bound form of the auxiliary protein rather than as a binding domain for the analyte itself. For example, auxiliary analyte binding proteins termed periplasmic binding proteins can act as primary sensing moieties that, in their ligand bound states, interact with the sensory moiety of a transmembrane MCP receptor to produce a response from the MCP signaling moiety. See, for example, Kondoh et al.(1979), Identification of a methyl-accepting chemotaxis protein for the ribose and galactose chemoreceptors of Escherichia coli, Proc Natl Acad Sci USA 76: 260-264, which is herein incorporated by reference in its entirety, particularly in parts pertinent to the use of auxiliary analyte binding proteins in accordance with various aspects and preferred embodiments of the present invention as discussed above and elsewhere herein.

The galactose and ribose periplasmic binding proteins from E. coli comprise sensing moieties useful in accordance with this and other aspects and preferred embodiments of the invention. When bound to cognate analyte ligand (galactose and ribose, respectively) each of these proteins binds to the N-terminal periplasmic sensing region of the Trg receptor, an MCP, and thereby act as effectors of E. coli chemotaxis.

In certain other preferred embodiments of the invention in this regard, a sensing moiety useful therein may be comprised in an auxiliary protein that is anchored or imbedded in the membrane. Such auxiliary membrane proteins can act as auxiliary proteins as described above; and, they also can serve as quasi receptors, akin to MCP, by interacting with transmembrane or other portions of MCP-like transmembrane proteins. Sensory rhodopsin, for example, undergoes a change in state when it is activated by light. It is an integral membrane protein, but, it does not directly effect a detectable signal. Instead, when activated, it impinges on HPT, an associated MCP-like protein, which is then stimulated to engender the production of a detectable signal. See, for example in this regard, Spudich (2002): Spotlight on receptor/transducer interaction, Nat Struct Biol. 9: 797-799, which is herein incorporated by reference in its entirety, particularly in parts pertinent to the use of auxiliary analyte binding proteins in accordance with various aspects and preferred embodiments of the present invention as discussed above and elsewhere herein.

The foregoing discussion and examples of auxiliary proteins are merely illustrative of various facets of the invention in this respect. Numerous other proteins of this type are known that can be used in accordance with the invention. Moreover, a variety of signaling moieties, modules and the like, can be utilized together with auxiliary proteins of the type discussed herein, and moieties for binding specifically to them, to make and use sensor elements employing auxiliary proteins in accordance with the invention.

Sensing Moieties

A sensing moiety in accordance with various aspects and preferred embodiments of the present invention is an entity that interacts with one or more analytes. Generally a sensing moiety interacts preferentially, selectively, specifically, substantially exclusively or exclusively with one specific analyte, a limited number of specific analytes or a specific group of analytes. A sensing moiety and the specific analyte or analytes with which it interacts are referred to as: cognates of one another, cognate sensing moiety and cognate analyte, cognate pair, etc.

In general the sensing moiety is in one state when interaction with cognate analyte is not taking place or is below a certain threshold level, and it is in a different state when interaction with cognate analyte meets or exceeds a certain threshold. Change in conditions that decrease interaction from above to below the threshold level or that increase interaction from below to above the threshold cause the state of the sensing moiety to change.

In typical sensors in accordance with various aspects and preferred embodiments of the invention herein disclosed, the state of the sensing moiety, or a change in the state, is transduced and thereby impinges on one or more signaling moieties (either alone or in combination with the transduction of states of other sensing moieties). As a result, the detectable signal engendered by the signaling moiety is influenced by and reflects the interaction of one or more sensing moieties with their cognate analytes.

A sensing moiety generally interacts with its cognate analyte or analytes through chemical interactions, predominantly; but, it may do so through physicochemical or physical interactions or a combination thereof. Generally, sensing moieties interact with analytes through non-covalent chemical interactions, but they also may interact through covalent bonding.

The interaction between a sensing moiety and cognate analyte may involve formation of a short lived transient analyte sensing moiety complex, a longer-lived transient complex, a stable complex, an essentially non-dissociating non-covalent complex or a covalently bonded complex, to name a few. The forward and back rates of the interactions vary greatly for different cognate sensing moieties and analytes.

In a given sensor in accordance with the invention, many sensing moieties are available to interact with cognate analytes, and production of a detectable signal reflects statistical parameters of the interactions of the plurality of available sensing moieties with cognate analytes in the “environment” to which the sensor is exposed.

Different sensor designs make use of different binding properties and kinetic characteristics of cognate pairs of sensing moieties and analytes and, moreover, use different combinations of sensing moieties to detect and/or measure one or more cognate analytes over narrow and/or broad ranges of occurrence, alone or in combination, reversible or irreversibly. As a result, the invention provides sensors that can match just about any type of analyte sensing requirement.

The analytes can be chemical in nature as described elsewhere herein, to such as ligands (including, for instance, chemosensory attractants, repellents) and ligand-binding auxiliary proteins, peptides, polypeptides, proteins, polynucleotides, cell surface markers, proteoglycans, carbohydrates, lipid adducts, or a wide variety of other compounds, substances, complexes, polymers and the like. The analytes also can be physical phenomena and/or properties, such as temperature, pressure, light, and motion, to name just a very few.

Structures of Sensing Moieties and Modules

Sensing moieties in accordance with the invention may be integral but most often are comprised within another entity. They may be formed of continuous or of discontinuous regions of the entity.

Sensing moieties can be comprised of one or more sensing domains, which, typically, are those continuous or discontinuous portions of individual subunits that together form a sensing moiety. A sensing module, as the term is used herein, comprises a sensing moiety and facilitates the interchange of the sensing moiety with different signaling moieties and/or other components.

The sensing moieties and domains of preferred embodiments can be based on and/or derived from binding and capture domains and/or entities that occur naturally. They also can be designed and synthesized de novo.

In certain of the preferred embodiments in accordance with various aspects of the invention in this regard the sensing moiety is or is comprised of one or more sensing domains of one or more polypeptides.

For instance, as an illustrative example, in certain particularly preferred embodiments of the invention in this regard, the sensing domain is a four helix bundle located toward the Nterminus of a polypeptide, and the sensing moiety is comprised of two four helix bundles associated with one another in a homodimer formed by two copies of the polypeptide.

Further illustrative in this regard, for instance, are certain highly particularly preferred embodiments of this type in which the four helix bundle domain is optimized by in vitro mutation and selection of a four helix bundle forming a polypeptide initially selected from a library of four helix bundle forming polypeptides synthesized de novo for its affinity, avidity and selectivity of binding the target analyte.

As mentioned elsewhere, in preferred embodiments in accordance with various aspects of the invention herein disclosed the sensing domains are continuous or discontinuous regions of one or more polypeptides. In certain of the particularly preferred embodiments in this regard the polypeptides and/or the regions thereof that form a sensing moiety and/or sensing domain(s) and/or sensing modules are one or more of chimeric polypeptides, proteins and chimeric proteins.

Sensing Moieties and Modules Based on Bacterial Proteins

Particularly preferred in this regard are polypeptide regions that are or that are derived from receptor polypeptides and/or proteins, especially chemotaxis receptor polypeptides and/or proteins, among which chimeric chemotaxis receptor polypeptide regions, polypeptides and or proteins are especially highly preferred, very especially polypeptides that are or that are derived from or that are close homologs of a methyl accepting bacterial chemotaxis receptor.

Among highly preferred embodiments of the invention in this regard are prokaryotic, particularly bacterial, receptor polypeptides and/or proteins, especially the periplasmic portion of such polypeptides and/or proteins. Particularly highly preferred in this regard are chemosensory receptor polypeptides and/or proteins, especially chemotaxis receptor polypeptides and/or proteins of motile prokaryotes, especially bacteria. Very particularly highly preferred in this regard are chemotaxis receptor polypeptide that undergo adaptive modification, particularly those of bacterial, especially gram negative bacteria, very particularly E. coli and Salmonella. Further especially preferred in this regard are the methyl accepting chemotaxis receptor polypeptides and/or proteins of prokaryotes, particularly those of gram negative bacteria, especially those of E. coli and of S. enterica, especially S. enterica serovar Typhimurium, very particularly as to these the E. coli Tar, Tsr, Trg and Tap MCP receptors and/or polypeptides and/or proteins derived therefrom and the S. enterica serovar Typhimurium Tar, Tsr, Trg and Tcp MCP receptors and/or polypeptides and/or proteins derived therefrom.

It is to be appreciated that sensing and signaling moieties can be derived from MCPs and then used separately, as well as together with one another, in making hybrid proteins useful as sensor elements in accord with the invention. For instance, the N-terminal aspartate binding moiety of the E. coli aspartate receptor, Tar (an MCP), can be fused to the signaling moiety of the E. coli serine receptor, Tsr (also an MCP) to make a functional chimeric receptor with the sensing specificity of Tar and the signaling specificity of Tsr.

To accomplish this, the DNA encoding the Tar MCP and the DNA encoding the Tsr MCP are cleaved at one of several restriction sites located in the portion that encodes protein sequences that are C-terminal to the second transmembrane sequence. The 3′ fragment of the Tar gene is then ligated to the 5′ fragment of the Tsr gene to produce a hybrid gene. This DNA sequence is then fused behind an efficient promoter such as lac within a high copy plasmid. The fused receptor product is then produced by cells exposed to the inducer, isopropylthiogalactoside. The resulting novel hybrid sensor elements can then be isolated by standard protein purification procedures, and used to form preferred embodiments of the invention. Well known methods that can be adapted to making hybrid proteins comprising sensing moieties in accordance with certain aspects and preferred embodiments of the invention in this regard are described by Krikos et al., Proc Natl Acad Sci USA 82:1326-1330 (1985) in their report on “Chimeric chemosensory transducers of Escherichia coli” which is incorporated by reference herein in its entirety in this regard particularly as to planning and performing the construction of hybrid MCPs useful in the invention.

It is to be appreciated that these and other receptor proteins can serve as the starting points or as scaffolds for making receptors comprising sensing moieties useful to sense just about any analyte or combination of analytes in accordance with the invention herein disclosed.

Sensing Moieties from Antibodies and Other Proteins of the Immune System

Chimeric receptors also can be made from the variable antigen binding domains of antibodies. With current technology it is possible to develop antibodies to most chemicals, determine the gene sequence of the antibody, and clone a single chain protein that includes the ligand-binding variable regions from both the light and heavy chains. See, for example Chadd and Chamow (2001) and/or Venturi et al., (2002), each of which is herein incorporated by reference in its entirety, particularly in parts pertinent to the use of antibodies and antibodyderived polypeptides and/or proteins in accordance with various aspects and preferred embodiments of the present invention in this respect.

For example, sensor elements can be made in accordance with this aspect of the invention by fusing the variable portion of an antibody gene with a gene for a Tar receptor such that the antibody gene fragment replaces the portion of the Tar gene encoding the periplasmic ligand binding domain, and the antibody variable region domain in the resulting hybrid polypeptide and/or protein functions to bind its cognate antigen and thereby undergo a change of state.

Sensing Moieties from Other Sources

Sensing domains and moieties in accordance with the invention are not limited to those mentioned above. They can be any of a wide variety of structures including those that are, are derived from, are related to, or are based on, for instance: a binding domain of a polypeptide of a cellular receptor, other than those involved in chemotaxis, particularly a binding domain of a polypeptide of a Type 1 cell surface receptor, the antigen binding domain of an antibody polypeptide or a T cell antigen-binding receptor, a binding domain of a periplasmic binding protein, a binding domain of an integral membrane protein, such as a 7-transmembrane Gprotein coupled receptor, and other binding domains discussed elsewhere herein.

De Novo Synthesis

Sensing domains, moieties and/or modules are made in accordance with certain aspects and preferred embodiments of the present invention by de novo synthesis. De novo synthesis in this regard can be, to mention just a few possibilities, total de novo synthesis carried out without reference to other chemical structures that function as binding and/or sensing entities, de novo synthesis that is based on, designed around, informed by and/or takes as its starting point a structure or structures and/or one or more portions of a structure or structures known to function as binding and/or sensing entities.

In certain particularly preferred embodiments of the invention in this respect, sensing domains, moieties and/or modules are made by de novo synthesis based on chemosensory proteins of bacterial, in particular, methylated chemosensory proteins, especially the methylated chemotaxis receptor proteins of bacterial.

In preferred embodiments in a related aspect of the invention in this regard, sensing domains, moieties and/or modules are made by de novo synthesis based on four-helix binding domains, particularly the four helix binding domains found in some chemosensory proteins of motile bacteria, especially the four helix binding domains of methylated bacterial chemosensory proteins.

For instance, the E. coli and Salmonella Tar proteins both sense aspartate by directly binding to it in the periplasm. However, there is much wider divergence in the periplasmic sensing domain (only 67% sequence identity) than in the cytoplasmic domain (90% identity), even though the periplasmic domains of these two proteins have the same structure as determined by x-ray crystallography (Björkman et al., 2001). It appears that only a few critical residues form the aspartate binding site (Björkman et al., 2001) and, therefore, any four-helix bundle of the right shape that includes these residues in the critical positions should serve reasonably well as an aspartate receptor.

Kamtekar et al., 1993; Rojas et al., 1997; Moffet et al., 2001 describe methods for generating libraries of de novo proteins that fold into a four-helix bundle with a topology very similar to that of the Tar sensing domain, and are incorporated herein by reference in their entirety in parts pertinent to making the libraries, screening them to identify proteins of interest and related methods in this regard that can be useful in carrying out the same. Selection can be made from a library of this type to find proteins that bind a specific ligand (such as a heme group) using methods described in Rojas et al., 1997 and in Moffet et al., 2001, which are incorporated herein by reference in their entirety, particularly in this regard, as to methods for generating, screening, identifying and further producing libraries that encode and can be used to make four helix bundle domain-containing proteins that comprise those that bind a target analyte and/or ligand of interest.

As described by Kamtekar et al., (1993), for instance, a library of four-helix bundles is generated using solid phase DNA synthesis. At each position in the gene a single nucleotide or a random mixture of nucleotides is incorporated in order to, on average, generate a pattern of amino acid hydrophobicity that leads to the desired four-helix bundle In one synthesis reaction each DNA molecule generated should be unique, giving a theoretical library of about 10¹⁶ possible receptor sensing domains. From this theoretical library, about 10⁹ unique sequences can be recovered and screened in the laboratory. By taking this approach, a much larger volume of sequence space can be sampled and screened than by introducing mutations a few at a time into a naturally occurring receptor sequence.

Libraries of candidate sensing domains made in this way can be inserted into cloning cassettes and/or then into vectors so that they are disposed to function as sensor elements. The library then generally is transformed into a host in which the sensor elements can be screened for their ability to support responsivity of the host to the analyte of interest.

Target analyte binding to the transformed cells containing library vectors also can be assayed by panning techniques and/or by retention assays.

Cells that show desirable analyte binding properties can be cloned and expanded and then further characterized. DNAs encoding the sensing moieties can be isolated from the best candidates and further developed by mutation and selection, by directed evolution and by other techniques to provide optimized sensing moieties.

Directed Evolution

Directed evolution can be used to obtain sensing moieties that interact with target analytes with enhanced affinity and specificity. The process of directed evolution uses in vitro random mutagenesis and recombination in order to incrementally develop a protein with improved properties. After each round of mutagenesis or recombination the new proteins are screened. Genes encoding improved proteins are retained in the population while the others are discarded. Methods for directed evolution are described in, for instance, Moore et al., (1997) and in Zhao and Arnold (1997) which are herein incorporated by reference in their entireties in parts pertinent to directed evolution.

Signaling, Signaling Moieties, Modules

A variety of signaling moieties can be used in accordance with the invention herein described. In preferred embodiments, signaling moieties are derived from signal generating proteins and/or polypeptides of cells. Particularly preferred in this regard are signaling moieties of MCPs, particularly those of E. coli, especially for instance, of E. coli tar.

The system output that is mediated by CheY phosphorylation is quite flexible. There are numerous different homologous response regulators in bacteria (the E. coli genome encodes over 30 different CheY homologs, most of which function to regulate gene expression) (Grebe and Stock, 1999), and it has previously been shown that a chimeric protein formed by joining the chemotaxis response regulator, CheY, to a transcription factor can be used to obtain a change in gene expression in response to CheA signaling (Allen et al., 2001).

The chemotaxis response thus can be coupled to an easily detectable output such as the production of light through the expression of a gene that encodes luciferase.

An output that relies on a change in gene expression requires several minutes to produce a measurable response, however, and the response persists in the absence of a signal until the gene product is degraded. Thus, transcriptional outputs essentially integrate signals over time. This might be useful for screening cells for a desired receptor, or in cases where an immediate readout is not needed. However, for many types of stimuli a rapid, real-time response would be desirable. The chemotaxis system can provide such a response, since cells normally respond to chemical signals within 0.1 sec, and it is possible to couple the CheY phosphorylation to an immediate fluorescence output. This has been done using fluorescence resonance energy transfer (FRET) between the cyan and yellow derivatives of the green fluorescent protein (GFP) fused to CheY and to a protein, CheZ, that specifically binds to phospho-CheY (Sourjik and Berg, 2001).

Coupling Regimes

A number of different types of signaling regime can be embodied in accordance with the present invention. Among these are the following.

Qualitative regimes in which a detectable signal or determinable change therein indicates the presence of the target analyte in a concentration above a threshold minimum concentration associated with the signal that just provides the minimum acceptable S/N level.

Static quantitative measurement regimes in which signal intensity or frequency indicates the amount of the analyte at a given time—or integrated and/or averaged over a longer time period.

Dynamic measurement regimes in which the signal indicates the difference between two points in a on-going time series.

Relative dynamic regimes in which the quantitative aspect of the signal indicates the difference between two points in the time series relative to the ambient condition at the start or the end point.

Linear regimes in which the quantitative aspect of the signal varies directly with the quantitative measure of the target.

Non-linear regimes in which the quantitative aspect of the signal depends in a definite but non-linear way on the quantitative measure of the target.

Dynamic regimes in which the signal, or determinable variation therein, indicates changes in the quantitative measure of target between the present measurement and a reference measurement or average or the like.

Those in which the relationship between the target level and the signal “quantity” is linear, is fixed fractional incremental (e.g. fixed percent), is varying fractional incremental (fractions that scale with the level), is log and/or exponential, and/or is differential.

Analytes

Sensors in accordance with the present invention can detect the absence or the presence, and/or to quantify the amount, concentration, flux, density or the like of a very wide, essentially unlimited range of chemical and physical analytes.

The analytes can be virtually anything, but typically are chemical or biological entities or both, for instance, including, among others, peptides, polypeptides, proteins, small organic molecules that bind peptides, polypeptides and/or proteins, ligands of other types, carbohydrates and polysaccharides, to name just a very few.

Moreover, sensors of the invention can detect and/or determine quantitatively the amount, concentration, flux, density or the like not only of one but of more than one target analyte in a single sensor and/or simultaneously. Analytes can be sensed in accordance with the invention, inter alia, directly by chemical interaction, such as ligands binding to cognate receptors, by physical interaction—such as light-activation of sensory rhodopsin, by indirect actions, e.g. ligand biding to auxiliary protein(s).

Cell Free and Purified Sensors

Cell free sensor systems, including sensor systems made of purified components, and purified components for sensors of the present invention can be made in accordance with the present invention using cell free extracts that reconstitute bacterial chemosensory systems and system components in vitro, from purified chemotaxis membrane vesicles and from purified chemotaxis proteins.

Methods useful for carrying out fractionations, purifications and reconstitutions in accordance with these aspects and embodiments of the invention are described by Ninfa et al., 1991 and Levit et al., 2002, which are incorporated herein by reference in their entireties, particularly in parts pertinent to making components and proteins and the like for use in cell free sensors of the present invention.

It will be appreciated that cell free systems in accordance with the invention in this regard can incorporate components from a variety of sources and may utilize compositions, compounds and/or components derived from cells that are incomplete in some way, such as missing or depleted in one or more sensor components, including those that would occur in a wild type cell. The missing components added to complete the cell-derived constituents of the sensor in order to reconstitute a functional sensor element and/or sensor can be any of the moieties or modules.

Discussion of a Few Exemplary Modular Sensor Elements

In this regard the invention relates in a particularly simple embodiment of this type to modular sensors comprising one of a first plurality of interchangeable sensing modules and one of a second plurality of interchangeable signaling modules, wherein the sensing module comprises a sensing moiety that has two states, interacts specifically with a target analyte, is in one state when the target analyte is below a threshold level and is in the other state when the target analyte is above the threshold level; wherein further the signaling module comprises a signaling moiety that engenders the production of a detectable signal indicative of the state of the sensing moiety and/or a change thereof.

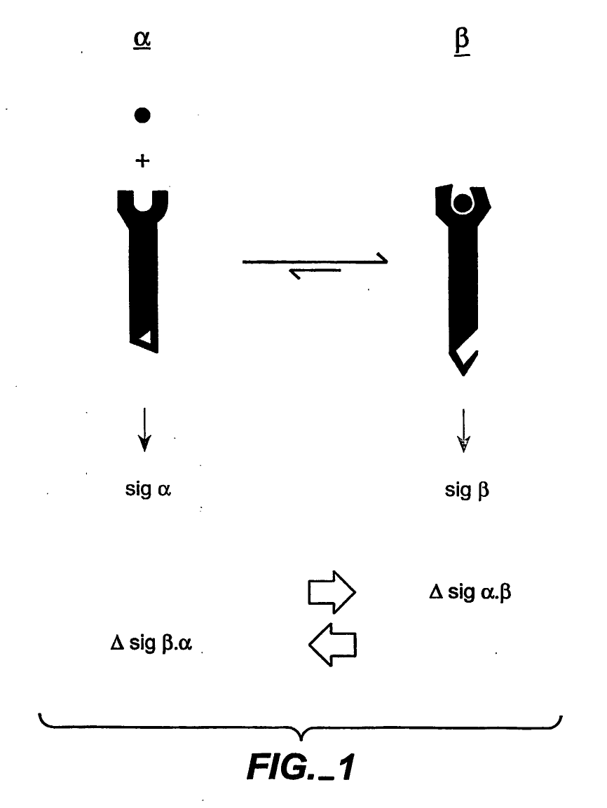

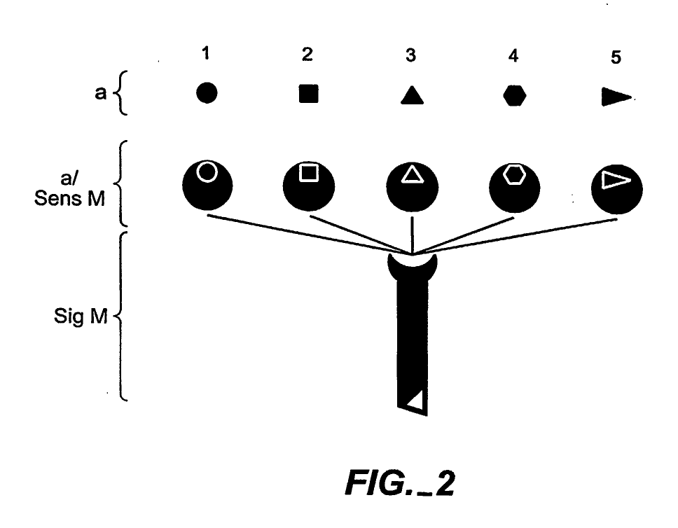

Elemental sensors in this regard are illustrated in FIGS. 1 and 2. FIG. 1 shows a simple one element modular sensor under conditions α and β. The sensor is depicted as a Y. The top of the Y represents the sensing module, and the sensing moiety, figuratively, is depicted by the inside surface of the top V. The signaling moiety is depicted by the triangle at the Y's bottom, closed on the left, open on the right. The target analyte is represented by the closed circle.

On the left, under the conditions of α, the target analyte is below the threshold level and, therefore, it is depicted away from the sensor element. The signaling module produces a first signal “Sig α” under these conditions, indicative of the below-threshold level of the analyte. On the right, under the conditions of β, the target analyte is above the threshold level and it is depicted within the pocket of the Y, interacting with the sensing moiety. The signaling module produces a second signal “Sig β” under these conditions, indicating that the analyte is at or above the threshold level.

The difference in the signal engendered by the signaling moiety when conditions change from α to β is noted on the right as, Δsig α-β, and when they change from β to α as Δsig β-α on the left.

In certain preferred embodiments of the invention discussed in greater detail below, the sensor element is a receptor, in certain of the particularly preferred embodiments of the invention in this regard, it is an MCP receptor, especially an MCP receptor homologous in structure and function to an MCP receptor of the type illustrated on the left in FIG. 11, or an analog, variant, mutant or mutein thereof.

Further, in another related aspect, certain preferred embodiments of the invention provide sensors comprising a plurality of sensor elements that switch states at different thresholds, the signaling moieties independently engender additive signals indicative of the states of the sensing moieties, and the strength of the resulting overall detectable signal is quantitatively indicative of the level of the ambient target analyte and, moreover, varies directly with variation in the level of the analyte.

The interchangeable modularity of the sensing modules and the sensing moieties is depicted schematically in FIG. 2. The sensor element is depicted in FIG. 2 as a top-truncated Y with a triangle at its base (SigM). The triangle represents the signaling module. The top truncation represents the attachment site for sensing modules. Five different analytes (a) are depicted by geometrical shapes numbered 1 through 5 across the top of the figure. The five analytes also are shown bound to five different analyte-specific sensing moieties in five sensing modules (a/SensM), aligned across the figure just below the analytes. Finally, the figure indicates by the lines from each sensing module to the truncated sensor element that each of the five sensing modules can be attached to the same sensor element comprising the same single signaling moiety.

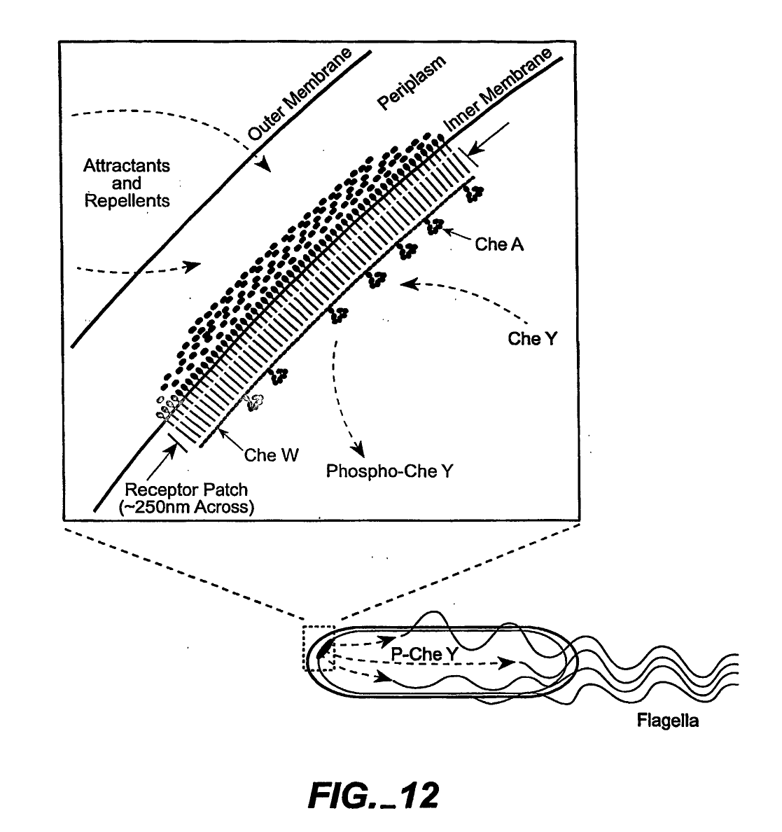

The efficiency with which the state of the sensing moiety is transduced to the signaling moiety and thence with which the signaling moiety engenders a detectable signal differs between receptors. Interchangeable modularity in this regard is further illustrated in FIG. 11 by the schematic depiction of the conserved modular structure of prokaryotic chemotaxis receptor proteins. These membrane spanning proteins typically are homodimers composed of four regions (modules): in FIG. 12 labeled “Sensing Domain,” “Transmembrane Helices,” “Linker Region” and “Cytoplasmic Domain.”

The Sensing Domains of different receptor proteins can be interchanged and still provide functional receptors, and in this regard exemplify interchangeable sensing modules in accordance with the invention. The Sensing Domain of each chemotaxis receptor interacts with specific attractants and/or repellents and the state of interaction of the Sensing Domain to attractant or repellent is transduced to the Cytoplasmic Domain and therein impinges oil the region labeled “CheA/CheW Binding” in the figure. This region interacts with Chew and CheA, modulates CheA kinase activity and, thereby, modulates CheA auto-phosphorylation and phosphorylation of CheY. As a result, it modulates tumbling frequency. The Cytoplasmic Domains of different chemotaxis receptors can be interchanged, and the CheA/CheW Binding region is exemplary of interchangeable signaling modules and signaling moieties in accordance with the invention.

It is to be appreciated, by way of further illustration, that the change in CheA kinase activity can be detected and/or determined and thus can serve as a detectable signal indicative of the state of the sensing moiety in accordance with the invention. Likewise, a substrate for CheA kinase in these embodiments can provide a detectable signal indicative of the state of the sensing moiety and, thereby the presence of the target analyte above or below a threshold or quantitatively by increments or continuously.

In a particularly preferred embodiment in this regard, the effect of CheA kinase activity on CheY and CheZ provides a detectable signal that rapidly indicates the state of the sensing moiety. In a particularly highly preferred embodiment in this regard, FRET is used to generate the detectable signal. In an illustrative example of this, CheY is supplemented or replaced by a functional fusion of CheY to one or the other of a cyan and a yellow derivative of the green fluorescent protein (“GFP”). CheZ, likewise, is replaced or supplemented by a functional fusion of CheZ to the other GFP derivative. When CheA kinase activity is low, there is little phospho-CheY. Since CheZ only associates with phospho-CheY, under these conditions, the two GFP derivatives of the fusion proteins are far apart, on average. As a result, there is little or no FRET from one to the other. When CheA kinase is active, however, CheY is phosphorylated, CheZ binds to the phospho-CheY, the two GFP derivatives in the two fusion proteins come into close proximity, and there is strong FRET to the acceptor GFP derivative when the donor GFP derivative is excited. It will be appreciated that the foregoing preferred embodiment can provide a continuously variable detectable signal indicative of the interaction of the sensing moiety with the target analyte, insofar as CheA kinase activity and the effect of the signaling moiety thereon both are continuously variable.

A Few Configurations of Sensor Elements and Sensors

Two Sensor Elements for a Single Target Analyte

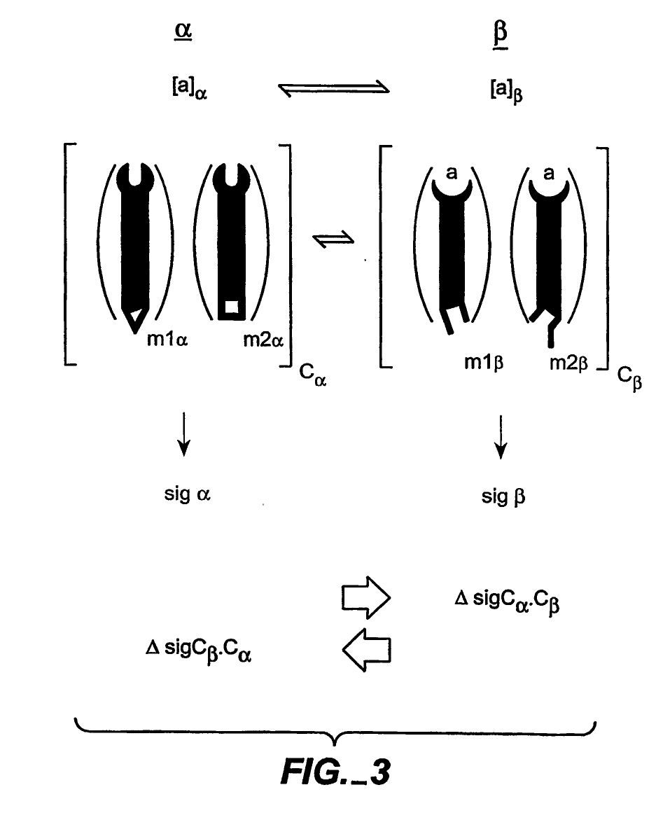

In certain aspects of the invention, certain of the preferred embodiments provide sensors comprising two different sensor elements having the same sensing moiety with different signaling moieties. A sensor of this type is depicted schematically in FIG. 3, which shows a sensor (square brackets) containing a pair of receptors (sensor elements) exposed to two different conditions, α and β. Two Y shaped objects represent two different receptors. The brackets indicate that the receptor pair function in a unit together. On the left the pair is exposed to condition α, characterized by analyte target concentration α. On the right the same pair of receptors is exposed to condition β, characterized by target concentration β. The sensor is depicted as a Y. The top of the Y represents the sensing module, and the sensing moiety, figuratively, is depicted by the inside surface of the pocket. The signaling moiety is depicted by the triangle at the Y's bottom, closed on the left, open on the right. On the left, under the conditions of a, the target analyte is below the threshold level and, therefore, it is depicted away from the sensor element. The signaling module produces a first signal “Sig α” under these conditions, indicative of the below-threshold level of the analyte. On the right, under the conditions of β, the target analyte is above the threshold level and it is depicted within the pocket of the Y, interacting with the sensing moiety. The signaling modules produce a second signal “Sig β” under these conditions, indicating that the analyte is at or above the threshold level.

The difference in the signal engendered by the signaling moiety when conditions change from α to β is noted on the right as Δsig-β and when they change from P to α a Δsig-αon the left.

Multiple Sensor Elements Specific to Multiple Target Analytes

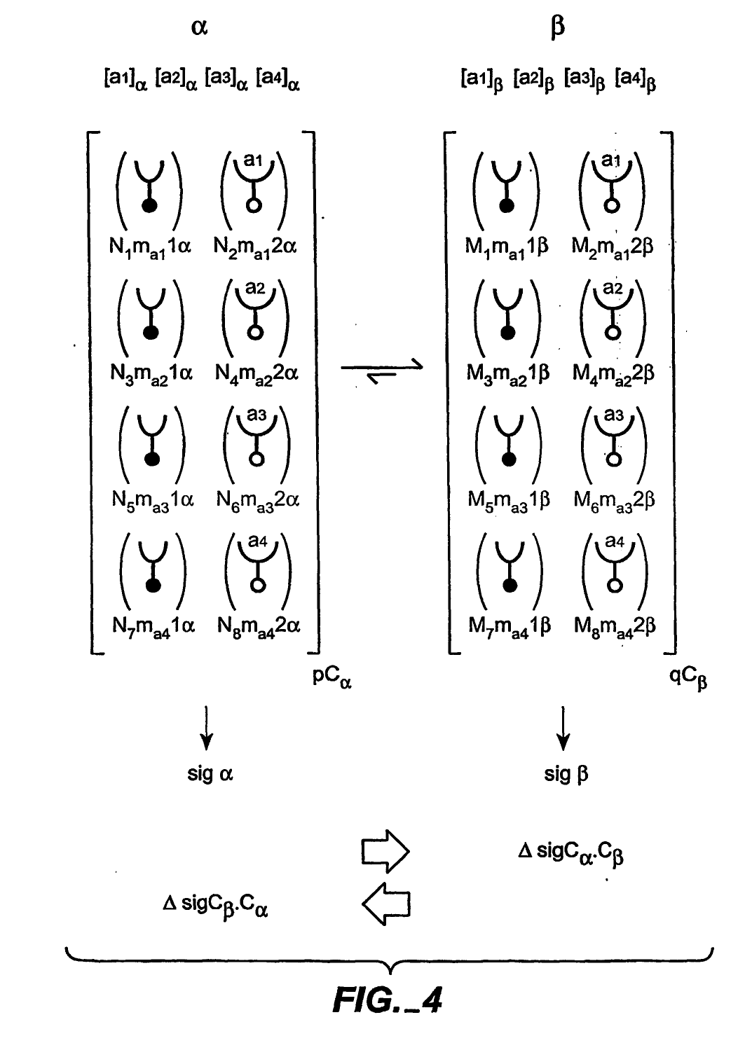

In certain aspects of the invention, certain of the preferred embodiments provide sensors comprising different sensing moieties specific for different analytes. A sensor of this type is depicted schematically in FIG. 4. The Figure provides a general description of signaling from complexes made up of four different sensor elements each specific for a different analyte. Each of the sensor elements has two states. In one state analyte is not bound and the state is depicted as a closed circle. In the other state specific analyte is bound and the state is depicted as an open circle. The sensor is depicted under two conditions, α and β. Under condition α the four specific analytes, a1, a2, a3, a4, are present in respective concentrations designated by subscript α. The concentration of the analytes under condition β is designated by subscript β. Each element is designated by “m” and a subscript indicating its analyte specificity followed by a 1 or a 2 indicating its state. Under condition α there are -1 through -8 instances of each element in each state, respectively. Under condition β there are -1 through -8 instances of each element-state, respectively. Combinations of the elements in one state or the other act collectively in a complex designated by the brackets. There are p complexes formed under condition a (PC). There are q complexes formed under condition β. Collectively these complexes give rise to a signal, SigM, under conditions a and a signal, SigM, under conditions β. When conditions change from α to β the change in the signal is designated Δsigc_{α}c_{β}. When conditions change from β to α, the change in the signal from sigM to sigM is designated Δsigc_{β}c_{α}.

It will be appreciated that the FIG. 4, while providing a general description of a four receptor system specific for four analytes, is illustrative, not exhaustive or exclusive. As illustrated elsewhere herein, there can be more than one element for a particular analyte. Furthermore, there can be more than four analytes, more than four elements, more than two states, more than eight element-states, and more than one signal under a given condition in sensors according to the invention. Furthermore, it will be appreciated that the number of instances of a given element-state may be zero in a given complex or may be the same under two given conditions such as α and β. Moreover, the makeup of the complexes, Cα, that contribute to sigM do not all necessarily have the same composition of elements-states. Various element-state combinations may be sufficient for a complex to engender the production of a signal.

Several Sensor Elements for One Target Analyte

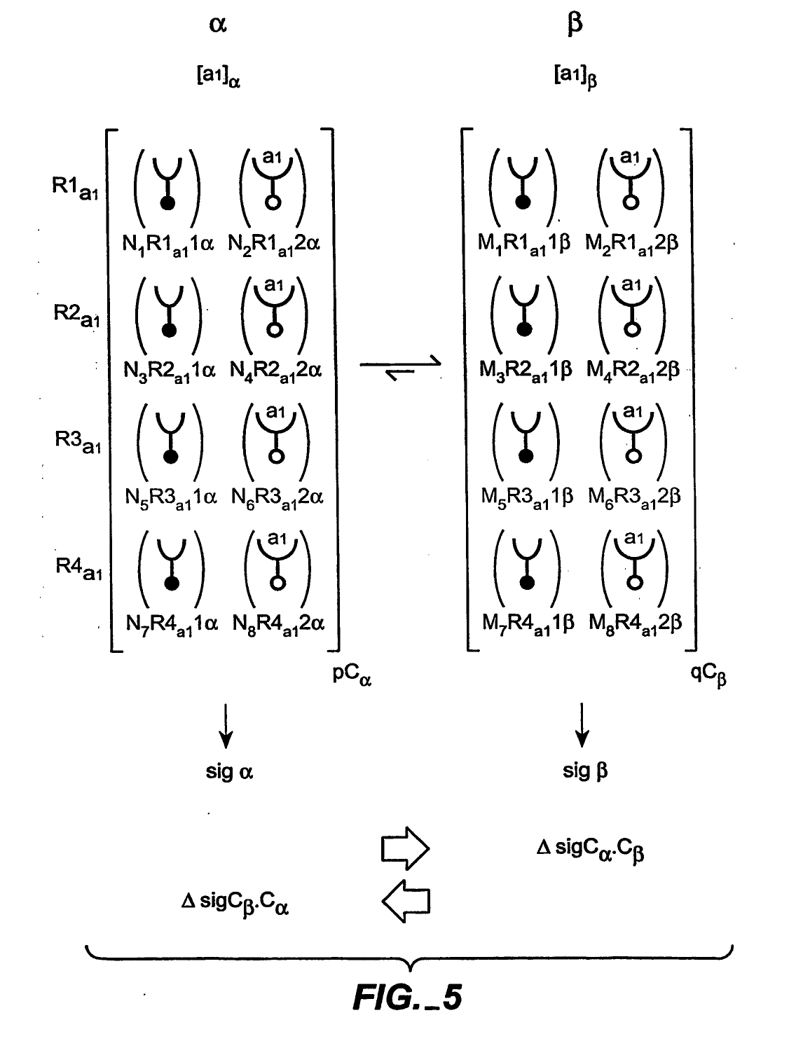

In certain additional aspects of the invention, certain of the preferred embodiments provide sensors comprising several different sensing elements having different sensing moieties specific for the same analyte. FIG. 5 depicts an illustrative example in this regard. The Figure provides a general description of a sensor comprised of four different sensor elements R1ₐ₁R4_{α1}. Each element has two states, 1 and 2, indicated by a closed circle and an open circle, respectively. There are N₁ through N₈ instances of each of the eight possible sensor element-states, in each of the β complexes, Ca formed under condition α that give rise to the signal designated sigM. Under condition β, the number of instances of each of the eight sensor element-states is designated M₁ through M₈. In each of the q complexes, Cβ, that give rise to the signal designated sigM. The change in the signal going from condition α to condition β is designated Δsigc_{α}c_{β}. The change in the signal going from condition β to condition α is designated Δsigc_{β}c₆₀ .

Multiple Sensor Elements for Multiple Target Analytes

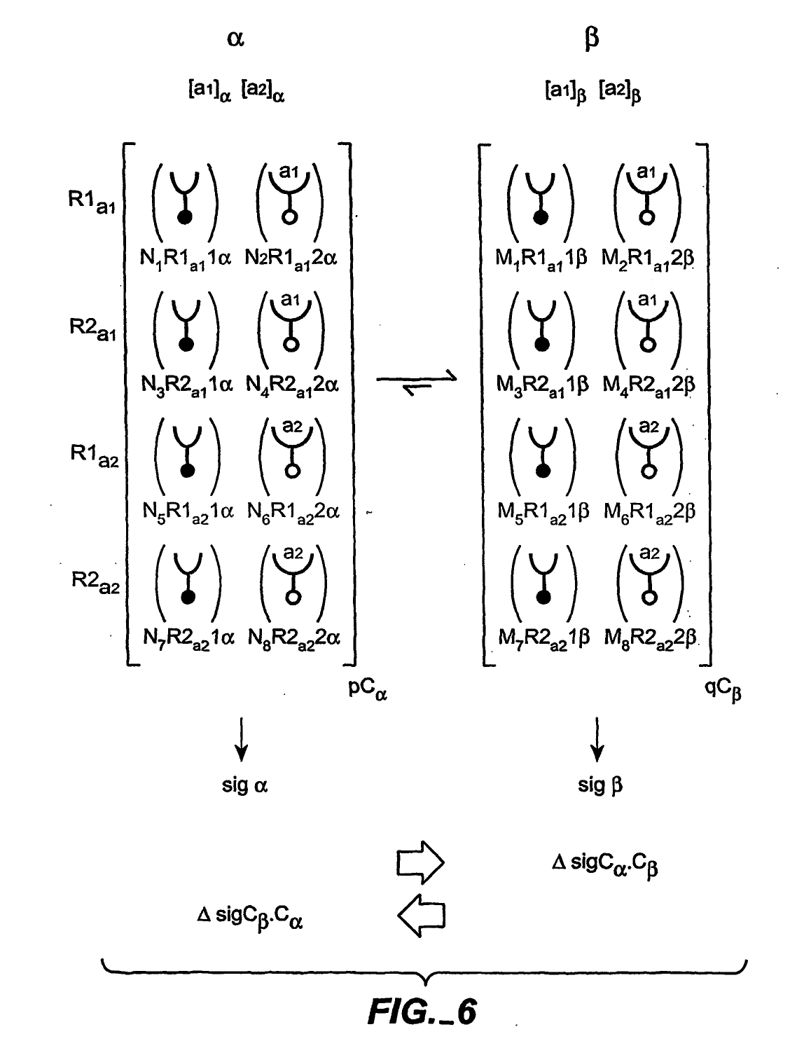

Certain particularly preferred embodiments of the invention relate to sensors comprising a multiplicity of sensor elements specific for multiple target analytes. FIG. 6 provides a general description of a sensor in accordance with the invention comprising complexes that can be formed by four sensor elements, two of which are specific for a first target analyte (a1) and two others that are specific for a second target analyte (a2). Each of the four sensor elements has two states. In the first state, depicted by the closed circle, the sensor element is not bound to its target analyte. In the second state, depicted by the open circle, target analyte is bound to the sensor-element. Each complex is comprised of an ensemble of sensor element-states. The four different two-state sensor elements define eight possible sensor-states. The number of instances of each sensor-state in any given complex (C) is denoted by N, through N, for complexes on the left and M₁ through M₈ for the complexes on the right. Under a first condition designated a on the left, the target analytes are present at first concentrations designated by the subscript α. Complexes are formed under these conditions, as well, made up of an ensemble of the sensor element-states. There are M₁ through M₈ instances of each of the eight possible sensor element-states, respectively, in each of the (q) complexes (C) formed under condition β, where q is the number of complexes. Taken together the complexes engender the production of a detectable signal designated sigM. As in previous Figures, the changes in the signals going from one condition to the other are denoted at the bottom.

Adaptative Sensor Elements

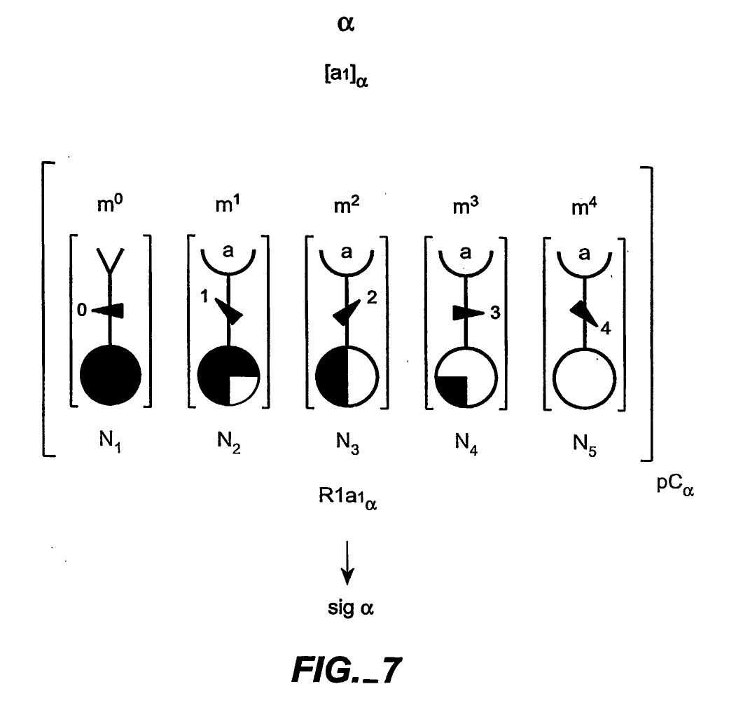

Another aspect of the invention relates in certain of its preferred embodiments to sensors with more than two states, particularly adaptive sensors. A five state sensor element illustrative in this regard is depicted in FIG. 7. In a first state designated “0,” the sensor element is not bound to target analyte, represented by the closed circle. In a second state, the sensor element is bound to target and engenders the production of a detectable signal at a first given level designated “1,” represented as a quarter-open circle. in a third state, the sensor element is bound to target analyte and acts to engender the production of a detectable signal at a second level designated “2,” represented as a half-open circle. In a fourth state, the sensor element is bound to target analyte and acts to engender the production of a detectable signal at a third level designated “3,” represented as a three-quarters open circle. In a fifth state, the sensor element is bound to target analyte and acts to engender the production of a detectable signal at a fourth level designated “4,” represented as a completely open circle. The levels represent gain between target binding and the action of the sensor element in engendering the production of a detectable signal. For example, in certain embodiments at level 0, when there is no target analyte bound to the sensor element, the sensor element may have no action leading to the production of a detectable signal; i.e., the sensor element is inactive in this regard. At levels 1 through 4, the sensor element is bound to the target analyte in this example and has increasing activity in engendering the production of a signal. The activity is weakest at level 1 and maximal at level 4. As depicted by the openness of the circles in the illustration, the activity at levels 2 and 3 is intermediate, half maximal at 2, in this example, and seventy-five percent of the maximum at level 3, depicted by the open regions of the circle.

The range in the example and illustration are merely exemplary. A variety of other ranges both discontinuous and continuous, linear and non-linear are possible. Furthermore, the state of a sensor element in accordance with the invention may be defined by two or more different mechanisms each with several such levels superimposed on one another to define the particular state of the sensor element.

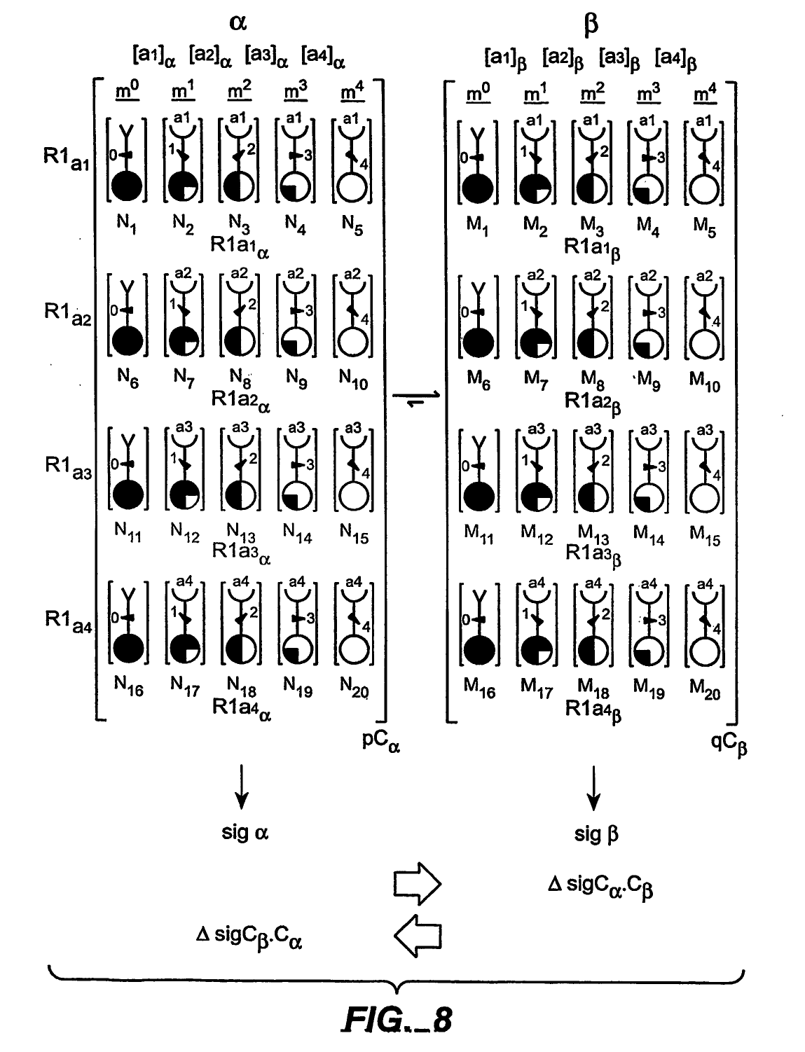

In accordance with the invention in this regard, further preferred embodiments relate to variable gain multiple state sensor elements as described immediately above in which the level of the sensor element, and thus the gain between binding and signal generating activity, changes in response to changes in the environment impinging on the sensor. In particularly preferred aspects and embodiments in this regard, the gain is modulated by the ambient level (concentration) of the target analyte. In especially particularly preferred embodiments in this regard, the gain is modulated so that it is highest in the absence of analyte, lowest when the level of analyte approaches saturation and scales to intermediate levels commensurate with intermediate levels of the analyte. Some embodiments of multiple state and multiple state adaptive sensors of this type are illustrated in FIGS. 8, 9, and 10.

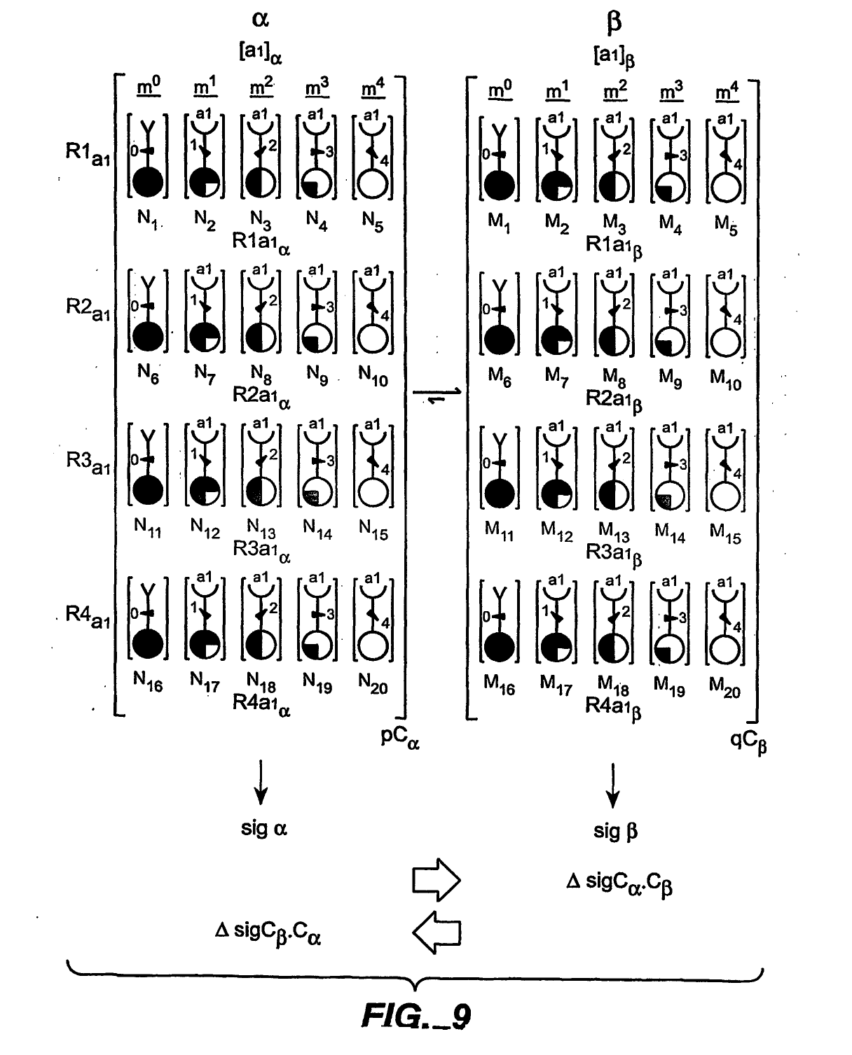

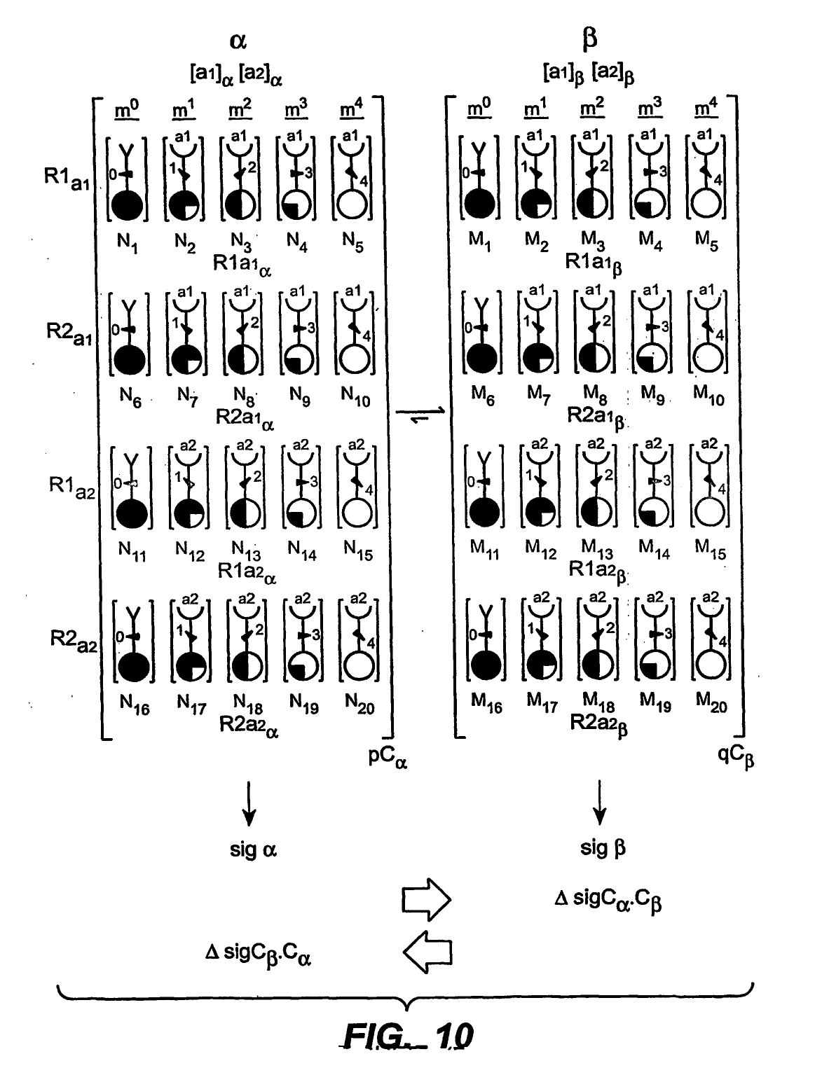

FIG. 8 depicts a four sensor element/four target analyte sensor in which each of the four different receptors has five states, as described for FIG. 7. The complexes formed and signals generated in the illustrated sensor are much as described in FIG. 4, except the sensor elements here have five states, instead of two. Accordingly, the complexes of this sensor comprise ensembles of the twenty sensor-states possible for four different five-state sensor elements. Under the conditions on the left, p complexes give rise to sigα, while on the right q complexes give rise to sigβ, Each of the complexes contributing to the total signal a on the left is the result of the composite activities of N₁ through N₂₀ instances, respectively, of each of the twenty possible sensor element-states. The same is true for the signal generated under. P on the right, but here the number of instances of each sensor element-state in a given complex is M₁ through M₂₀, respectively, for the twenty possible sensor element-states. The difference in the signals going from the first condition or the second, or the other way, is depicted as in previous Figures by the delta terms at the bottom of the illustration. FIG. 9 depicts a sensor comprising four different five-state sensor elements specific for the same single target analyte. The resultant signals and the differences in the signals going from one condition to the other are depicted in the same manner as for FIG. 8. FIG. 10 depicts a sensor comprising four different five-state sensor elements specific for two different target analytes. The sensor is much the same as that depicted in FIG. 6, except here the sensor elements have five states, whereas those in FIG. 6 have only two states and the complexes here are ensembles of instances of the twenty possible sensor element-states, whereas the complexes in FIG. 6 are ensembles of eight sensor element-states. It will be appreciated that FIGS. 7, 8, 9, and 10 are illustrative only of relatively simple types of embodiments involving just a few different receptors and/or target analytes. The invention, nonetheless, relates to sensors that are more complex than those depicted in these simple diagrams. Thus, in some preferred embodiments of the invention, there are many different receptors specific for many different target analytes. There may be many different levels of gain for some or all of the many different receptors in complex sensors according to the invention. Different sensor elements for the same target analyte can have different series of states, and the states for different sensor elements may be controlled by different mechanisms. Sensors in accordance with the invention can be comprised of hundreds, thousands, or millions of instances of sensor elements comprised in complexes that engender and/or effectuate the production of detectable signals indicative of the presence, the absence, the amount, changes in the amount, changes in the acceleration of change in the amount, and/or combinations thereof.

### EXAMPLES

The present invention provides for rapid development of novel biological sensors for almost any molecule of interest. Several ways of carrying out various aspects of the invention in accordance with certain of the preferred embodiments are illustrated by the particular examples set out below. There are many ways other than those set out in the examples to carry out the invention herein disclosed. In fact, the examples below involve approaches based only on bacterial MCPs and not other systems that would work as well. The focus on bacterial MCPs in the specific examples herein below does not reflect any limitation of the present to these proteins. Rather the focus is a matter of convenience and stems partly from the convenience of the E. coli system, and its ease of use, and partly from the usually detailed information about the system that is available.

Bacterial Chemotaxis

This summary provides, by way of introduction to the examples below, a brief overview of bacterial chemotaxis, in particular in E. coli, which highlights some advantages of these systems for sensors of the present invention.

Bacteria move by a variety of species-specific mechanisms involving numerous different types of flagella as well as by flagella-independent processes such as those responsible for gliding motility. Despite this diversity in motility, however, virtually all motile prokaryotes use the same basic regulatory apparatus to control their movements. See, for instance Stock and Surette (1996) and Falke (1997), which are incorporated herein by reference in their entireties in parts pertinent to using bacterial chemosensory and motility systems and components and aspects thereof in making and/or using and/or otherwise relating to sensors and other subject matter of the present invention.

Each bacterium generally has a single information processing organelle that generates signals to control motility in response to changing environmental conditions. These organelles are composed of a bundle of thousands of a-helical transmembrane receptor proteins that are organized together with an SH3-like adapter protein, CheW, and a protein kinase, CheA, as illustrated schematically in FIG. 12. The organization of the organelles is described in greater detail by Maddock and Shapiro (1993) and by Stock and Surette (1996) which are incorporated herein by reference in their entireties particularly in parts pertinent to using bacterial chemosensory and motility systems and components and aspects thereof in making and/or using and/or otherwise relating to sensors and other subject matter of the present invention.

Each prokaryotic chemosensory system has a number of different types of receptors distinguished by different N-terminal sensing domains that bind to different ligands. Except for the N-terminal binding site differences, the different types of receptors have the same structure. The domain structure of bacterial chemoreceptors, based on E. coli MCPs in particular, is illustrated in FIG. 11.

Despite their substantial structural differences, the sensory domains of all types of receptors are located on the outside surface of the cell membrane, where they are exposed to the external environment and can contact specific attractan(s) (such as nutrients like maltose or serine) repellent(s) (such as a toxin) to which the organism responds. The remainder of the receptor is located within or on the cytoplasmic surface of the membrane. In contrast to the differing structures and sequences of sensory domains, the receptors otherwise are very similar to one another in both sequence and structure, including the transmembrane domains and the regions that interact with CheW and CheA.

Attractant binding to the N-terminal sensing domains inhibits the protein kinase activity of CheA, whereas repellent stimuli activate the kinase. The CheA kinase mediates phosphorylation of the chemotaxis response regulator CheY. Phosphorylation induces a change in CheY structure that allows it to bind to a flagellar motor protein, and this binding causes a change in the direction a cell swims. Thus, increased concentrations of attractant inhibit CheA kinase activity, causing a drop in the level of phospho-CheY, and a concomitant decrease in the likelihood that the cell will change its direction of motion. Repellents have the opposite effect, causing an increase in CheA activity that leads to an increase in phospho-CAeY and an increased likelihood that the cell will change direction. The molecular processes of chemosensory systems are discussed, for instance, in Stock and Surette (1996) and in Falke et al., (1997), which are incorporated herein by reference in their entireties particularly in parts pertinent to using bacterial chemosensory and motility systems and components and aspects thereof in making and/or using and/or otherwise relating to sensors and other subject matter of the present invention.

Although the regulatory organelle in the chemotaxis systems of virtually all motile bacteria is comprised of the same three critical components (membrane bound receptors, CheW homologs and CheA homologs), motility exhibits a vast diversity among different types of bacteria. Each type responds to a unique set of metabolic and environmental factors, different from the other types. This great diversity in responsiveness in reflected in the extreme variability that is found in the sensory portions of the receptors, as revealed by examination of receptor sensory domain sequences from different motile bacteria.

Different bacteria vary widely in chemotactic response. Each type responds to a unique set of metabolic and environmental factors that, typically, is very different from that even of closely related bacteria. Altogether, bacteria display a vast, practically unlimited, sensory repertoire. The range of sensory ability is reflected in a correspondingly unusual variation in the receptor molecules of the chemosensory apparatus. However, the great variety of sensing by bacteria apparently is transduced by virtually all bacteria by just one particularly robust structure and mechanism, mediated by the three proteins found universally in bacterial chemosensory organelles: receptors, CheW homologs and CheA homologs.

In essence, the bacterial sensory apparatus is comprised of a variable module and a constant module. The variable module is comprised of the region of the sensory receptor protein that interacts with the environment and changes to reflect certain ambient conditions; e.g., concentration of a ligand. The variable module can interact in a reliable, repeatable, determinable way with just about any aspect of the environment, and its structure, accordingly, is highly variable. The constant module is comprised of the remainder of the receptor, one or more CheW homolog molecules, and one or more CheE homolog molecules. Changes that occur in the variable module in response to specific environmental factors are transduced by the constant module into an intracellular signal for regulating flagellar motion and, thereby, motility. Whereas, the variable module differs between bacteria in much the same way that they differ in chemotactic responses, the constant module is much the same for virtually all receptors, across all bacteria. See, for instance Levit et al. (2002), which is incorporated herein by reference in its entirety particularly in parts pertinent to using bacterial chemosensory and motility systems and components and aspects thereof in making and/or using and/or otherwise relating to sensors and other subject matter of the present invention.

For example, in E. coli, the five chemotaxis receptors have been investigated in detail. The variable N-terminal periplasmic sensory domains of four of the five E. coli chemotaxis receptors are four-helix bundles that either bind small molecule stimulatory ligands directly or interact with specific periplasmic binding proteins in their ligand-bound conformation. See Stock and Surette (1996) and Falke et al., (1997) each of which is incorporated herein by reference in its entirety, particularly in parts pertinent to using bacterial chemosensory and motility systems and components and aspects thereof in making and/or using and/or otherwise relating to sensors and other subject matter of the present invention. Although structurally similar, these sensing domains have a relatively low level of sequence conservation. The sensory domain of the fifth E. coli receptor is an unrelated PAS domain (Repik et al., 2000), and aside from very closely related species such as Salmonella, the receptor sensing domains of other bacteria appear to be completely unrelated to the four-helix bundle structure. Thus, the four-helix bundle is clearly a structure that can serve as a sensory domain for either direct ligand binding, or for binding of periplasmic binding proteins in their ligand bound state, but there is obviously not any tight constraint on the structure or sequence of the sensing domain.

Of particular importance, as to certain aspects and preferred embodiments of the present invention, is the prototypical modularity of prokaryotic chemosensory receptors, in which, perhaps most significantly, a practically unlimited variety and specificity of variable sensing domains function as interchangeable N-terminal variations atop an invariant pedestal, and the interaction of a very wide variety of sensory domains with a corresponding variety of ligand specificities all can work with the same transmembrane and cytoplasmic receptor domains in a functional chemosensory receptor that properly effectuates CheW-CheA-receptor signaling complexes in response to the ebb and flow of attractants and repellents in the environment, as discussed in greater detail in Wolanin and Stock (2002), which is incorporated herein by reference in its entirety particularly in parts pertinent to using bacterial chemosensory and motility systems and components and aspects thereof in making and/or using and/or otherwise relating to sensors and other subject matter of the present invention.

The modularity of sensory receptors can be engineered with sensory domains to detect virtually any signal or combination of signals that can physically interact with proteins including pH, metal ions, small molecule analytes, temperature, or light, among others. For instance, a chimeric of the light sensing region of Halobacterium salitnarum sensory rhodopsin fused to the constant region of an E. coli Tar receptor can confer phototaxis to E. coli, according to Jung et al., (2001), which is incorporated herein by reference in its entirety particularly in parts pertinent to using bacterial chemosensory and motility systems and components and aspects thereof in making and/or using and/or otherwise relating to sensors and other subject matter of the present invention.

The examples below demonstrate that it is possible to generate diversity in binding region domains, form functional receptor chimeras by fusing the domains to the constant region of a sensory receptor, and then utilizing the newly formulated receptor sensitivity in a sensor.

### Example 1

### E. coli eJSmcp1: lacl^{q}, tetR, che⁻, mcp⁻, phoB⁻ Host

E. coli is genetically altered so that it does not express chemotaxis receptor proteins; but, otherwise has a normal, functional chemotaxis system. The genes encoding the chernotaxis receptors are functionally or structurally deleted using well known recombinant DNA techniques. The receptor phenotype and receptor genotype of the lacl^{q}, tetR, che⁻, mcp⁻, phoB⁻, is confirmed by phenotypic analysis of receptor binding properties and by sequencing using specific primers. The lac, tet and phoB aspects of the strain also are verified by phenotypic analysis.

### Example 2

### pJStari1: Tar Inducible Plasmid Vector

A medium copy plasmid is constructed containing a Tar receptor gene operably linked to an inducible tightly regulated promoter (lacl) that can be controlled by the level of inducer in the growth medium. The Tar gene is engineered to contain convenient restriction sites in the plasmid near the beginning and the end of the region coding for the periplasmic sensing domain.

### Example 3

### pJSxcmcp1: CheA^{c}, CheW^{c}, CheY/PhoB¹, lacZ^{phoA} plasmid vector

A low copy number plasmid that expresses CheA and CheW constitutively, expresses a CheY-PhoB chimera under the control of a regulatable promoter, and expresses lacZ under the control of the phoA promoter is constructed as follows. CheA, CheW, CheY, PhoB and lacZ genes are cloned from E. coli by standard methods, or obtained from commercial, academic or other sources, and sequenced to confirm their identity. A standard plasmid vector is prepared for insertion of cassettes for expressing Che A, CheW, CheYIPhoB and lacZ. The Che A gene is ligated in operable linkage to a promoter that is expressed constitutively in E. coli. The Che W also is ligated in operable linkage to a promoter that is expressed constitutively in E. coli. DNAs encoding the phosphorylation domain of CheY (including the phosphate accepting site) and the transcription regulatory region of PhoB are operatively linked together to form a chimeric transcriptional regulatory protein that activates the PhoA promoter when it is phosphorylated, but does not activate the PhoA promoter when it is not phosphorylated. In an MCP competent cell in the presence of cognate ligand, CheA and Chew will engender phosphorylation, and consequent activation, of CheYIPhoB, which will activate transcription of lacZ and in the presence of calorimetric substrate, will result in the production of a colored product indicative of galactosidase activity.

### Example 4

### Induction of pJStari1 Tar expression in eJSmcp1

Competent eJSmcp1 cells are prepared by standard methods and then are transformed with pJStari1 plasmid DNA. Several colonies are isolated and cloned out, twice. A colony of one of the clones is isolated, characterized, expanded, re-characterized, aliquoted, labeled eJSmcpt1/pJStari1, and frozen for viability.

Aliquots of the cells are grown in the absence and presence of inducer. Host cells without pJStari1 are subjected to the same procedure using the same reagents at the same time. Aliquots of the transformed and wild type cells, independently, are grown in a concentration series of the inducer. The amount of induction in each culture is measured using a standard tar-specific protein assay. The amount of inducer necessary to produce in eJSmcp1/pStari1 the same amount of Tar as is found in wild type cells is determined using the same assay. These assay results are confirmed by titrating the amount of inducer necessary to restore swarming to aspartate in eJSmcp1/pStari1 to the levels seen in wild type cells.

### Example 5

### eJSmcp1[pJStari1, pJSxcmcp1]—eJSmcp1 Transformed with pJStari1 and pJSxcmcp1

eJSmcp1 cells are prepared for high-efficiency transformation using standard techniques, transformed with pJStar1 and pJSxcmcp1 and plated out to form individual colonies. Colonies of the appropriate phenotype are selected, are cloned out, and are rescreened for the primary phenotypic markers. Finally, the presence of the plasmids and their sequences are confirmed, phenotypic markers of expression are checked, and the levels of mcps, phoB, Lac and Pho are confirmed undetectable.

### Example 6

### me-D-aspartate Induces eJSmcp1 [pJStari1, pJSxcmcp1] Lac

eJSmcp1[pJStari1, pJSxcmcp1] is grown in the absence and in the presence of methyl-D-aspartate. Swarming is seen in the presence of the aspartate but not in its absence. The same results are seen in wild type aspartate responsive control cells, but not in eJSmcp1 cells that do not contain pJStari1 and pJSxamcp1.

### Example 7

### Luciferase Reporter System—pJSmcp-luc1

A plasmid, pJSmcp-luc1, is constructed the same as pJSxcmcp1, but with a luciferase gene (luc) under the control of the PhoA promoter instead of LacZ. When cells competent for the requisite MCP (such as cells transformed with pJStari1) are transformed with pJSmcp-luc1, and are exposed to the cognate analyte, luciferase is expressed, and a luminescent signal is produced when the cells are exposed to luciferase substrates and appropriate co-factors.

### Example 8

### FRET Reporter System—pJSmcp-FRET1 and 2

A plasmid, pJSmcp-FRET1 is constructed the same as pJScmcp1 but with a CheY-YFP chimera in place of the PhoB chimera and a CheZ-CFP chimera in place of lacZ. Cells containing this plasmid, that express the requisite chemosensory receptor, upon exposure to cognate analyte express CheY-YFP and, through the contact between CheY-YFP and CheZCFP produce a strong fluorescent signal when exposed to light of the appropriate wavelength. The FRET reporter system provides a high-speed readout.

### Example 9

### Four Helix Bundle Library Synthesis

A DNA library is synthesized encoding a library of four-helix bundle domain polypeptides predicted to be similar in size and folding to the Tar sensing domain. Synthesis is carried out so that the four helix bundle domains are comprised in a module (cassette) flanked by convenient restriction sites and/or sequence leaders for insertion into and recovery from a gene encoding an MCP in place of the sensing domain native thereto.

### Example 10

### Four Helix Bundle Library Insertion into pJStari1

The regions of DNA encoding the aspartate binding domains of the Tar receptor are removed from the Tar receptor DNA in pJStari1. The binding site deleted vector is transformed into a host, cloned out, verified by sequencing and then expanded. The binding domain deleted Tar encoding DNA is isolated from the cells, purified and then prepared for insertion of four helix bundle binding domains. The four helix binding domain-encoding DNA synthesized de novo is then ligated into the Tar DNA in place of the deleted aspartate binding site DNA. The resulting DNA library is transformed into eJSmcp1 and expressed. Chimeric Tar proteins thus produced are screened using the same techniques noted above for conditional expression of a marker dependent on activation by chemosensory responsiveness.

### Example 11

### In Vitro Assay for Direct Interaction of Receptors and Target Analytes

In addition to the foregoing assays, binding of wild type and chimeric receptors is determined by direct in vitro assay of complex formation by incubating analytes with receptor preparations or with cells under conditions for receptor-analyte binding and then detecting the complexes formed by standard sandwich assays or by labeling with the analyte or by other methods for the same well known to those skilled in the pertinent arts.

### Example 12

### Directed Evolution of Receptors for Improved Small-Molecule Binding

Candidate sensing moieties in chimeric MCPs such as those described above, are subjected to one or more rounds of directed evolution, including screening for and selecting those moieties with the most desirable properties by carrying out assays for the type discussed above.

### Example 13

### Antibody-Derived Library, Screening and Characterization

A DNA is isolated, cloned, sequenced and characterized that encodes the antigen-combining site (or sites) of one or more antibodies that have desirable binding properties for a target analyte. The DNA is subjected to in vitro mutagenesis. The antigen-combining regions of the antibodies in the library of variant DNAs produced by mutagenesis are inserted into an expression vector, operably linked to the open reading frame of an NH,-truncated deletion mutant of an MCP, so as to form the sensing domain of an intact MCP receptor. DNAs are transformed into a host cell and tested in the absence and presence of antigen. Responses to antigen of the antibody chimeric host cells are compared with those of wild type cells.

### REFERENCES

The following references are herein incorporated by reference in their entireties, particularly in parts pertinent to subject matter for which they are cited specifically elsewhere herein.
Allen, M. P., Zumbrennen, K. B., and McCleary, W. R (2001); Genetic evidence that the alpha5 helix of the receiver domain of PhoB is involved in interdomain interactions, J Bacterial 183, 2204-2211.
Björkman, A. M., Dunten, P., Sandgren, M. O., Dwarakanath, V. N., and Mowbray, S. L. (2001); Mutations that affect ligand binding to the E. coli aspartate receptor: implications for transmembrane signaling, J Biol Chem 276, 2808-2815.
Chadd, H. E., and Chamow, S. M. (2001); Therapeutic antibody expression technology, Curr Opin Biotechnol 12, 188-194.
Falke, J. J., Bass, R. B., Butler, S. L., Chervitz, S. A., and Danielson, M. A. (1997); The two-component signaling pathway of bacterial chemotaxis: a molecular view of signal transduction by receptors, kinases, and adaptation enzymes, Annu Rev Cell Dev Biol 13, 457-512.
Falke, J. J., and Hazelbauer, G. L. (2001); Transmembrane signaling in bacterial chemoreceptors, Trends Biochem Sci 26, 257-265.
Grebe, T. W., and Stock, J. B. (1999); The histidine protein kinase superfamily, Adv Microb Physiol 41, 139-227.
Jung, K. H., Spudich, E. N., Trivedi, V. D., and Spudich, J. L. (2001); An archaeal photosignal-transducing module mediates phototaxis in Escherichia coli, J Bacteriol 183, 6365-6371.
Kamtekar, S., Schiffer, J. M., Xiong, H., Babik, J. M., and Hecht, M. H. (1993); Protein design by binary patterning of polar and nonpolar amino acids, Science 262, 1680-1685.
Levit, M. N., Grebe, T. W., and Stock, J. 6. (2002); Dynamic Sensitivity in a Receptor-Kinase Signaling Array, submitted.
Maddock, J. R., and Shapiro, L. (1993); Polar location of the chemoreceptor complex in the Escherichia coli cell, Science 259, 1717-1723.
Mizuno, T., Mutoh, N., Panasenko, S. M., and Imae, Y. (1986); Acquisition of maltose chemotaxis in Salmonella typhimurium by the introduction of the Escherichia coli chemosensory transducer gene, J Bacteriol 165, 890-895.
Moffet, D. A., Case, M. A., House, J. C., Vogel, K., Williams, R. D., Spiro, T. G., McLendon, G. L., and Hecht, M. H. (2001); Carbon monoxide binding by de novo heme proteins derived from designed combinatorial libraries, J Am Chem Soc 123, 21 09-21 15.
Moore, J. C., Jin, H. M., Kuchner, O., and Arnold, F. H. (1997); Strategies for the in vitro evolution of protein function: enzyme evolution by random recombination of improved sequences, J Mol Biol 272, 336-347.
Ninfa, E. G., Stock, A., Mowbray, S., and Stock, J. (1991); Reconstitution of the bacterial chemotaxis signal transduction system from purified components, J Biol Chem 266, 9764-9770.
Quiocho, F. A., and Ledvina, P. S. (1996); Atomic structure and specificity of bacterial periplasmic receptors for active transport and chemotaxis: variation of common themes, Mol Microbiol 20, 17-25.
Repik, A., Rebbapragada, A., Johnson, M. S., Haznedar, J. O., Zhulin, I. B., and Taylor, B. L. (2000); PAS domain residues involved in signal transduction by the Aer redox sensor of Escherichia coli, Mol Microbiol 36, 806-816.
Rojas, N. R., Kamtekar, S., Simons, C. T., McLean, J. E., Vogel, K. M., Spiro, T. G., Farid, R. S., and Hecht, M. H. (1997); De novo heme proteins from designed combinatorial libraries, Protein Sci 6, 2512-2524.
Sourjik, V., and Berg, H. C. (2001); Receptor sensitivity in bacterial chemotaxis, Proc Natl Acad Sci USA 11, 11.
Stock, J. B., and Surette, M. (1996); Chemotaxis. In Escherichia coli and Salmonella typhimurium: Cellular and Molecular Biology, F. C. Neidhardt, ed. (Washington, D.C., ASM), pp. 1103-1129.
Stock, J. B. and Da Re (2000) ENCYCLOPEDIA OF MICROBIOLOGY Vol. 1, 2nd Ed., page 772, Academic Press (2000).
Venturi, M., Seifert, C., and Hunte, C. (2002); High level production of functional antibody fab fragments in an oxidizing bacterial cytoplasm, J Mol Biol 315, 1-8.
Wolanin, P. W., and Stock, J. B. (2002); Transmembrane signaling and the regulation of histidine kinase activity, In Histidine kinases in signal transduction, M. Inouye, and R. Dutta, eds. (New York, Academic Press); Zhao, H., and Arnold, F. H. (1997); Optimization of DNA shuffling for high fidelity recombination, Nucleic Acids Res 25, 1307-1308.

What is claimed is:

1. A sensor comprised of a sensor element comprising one or more sensing moieties and one or more signaling moieties, wherein each sensing moieties has one or more states indicative of a target analyte, one or more signaling moieties alone or in combination engenders the production of a detectable signal, the state or a change in the state of one or more sensing moieties is transduced to one or more signaling moieties whereby one or more of the signaling entities alone or in combination engender production of a detectable signal qualitatively or quantitatively indicative of the absence below a threshold, the presence above a threshold or the amount of a target analyte or of a change in the absence below a threshold, the presence above a threshold or the amount of a target analyte, and one or the other or both the sensing moiety and the signaling moiety is not a naturally occurring entity.

2. A sensor according to claim 1, wherein one or more sensing moieties binds one or more target analytes indirectly but not directly.

3. A sensor according to claim 1, wherein one or more sensing moieties is comprised in a sensing module and one or more signaling moieties is comprised in a signaling module, wherein the sensing module can be operatively linked to different signaling modules and the signaling molecule can be operatively linked to different sensing modules.

4. A sensor according to claim 1, wherein production of the detectable signal engenders changes in the sensor element whereby production of the detectable signal is terminated and a change in binding of the one or more target analytes to one or more sensing moieties is required to again engender production of a detectable signal.

5. A sensor according to claim 1, comprising a first plurality of sensing moieties that directly or indirectly bind a first target analyte and a second plurality of sensing moieties that directly or indirectly bind a second target analyte, further comprising one or more first signaling moieties that engender production of a first detectable signal and one or more second signaling moieties that engender production of a second detectable signal distinguishable from the first detectable signal, wherein the first detectable signal is qualitatively or quantitatively indicative of the absence below a threshold, the presence above a threshold, or the amount of the first target analyte or of a change in the absence below a threshold, the presence above a threshold, or the amount of the first target analyte, and the second detectable signal is qualitatively or quantitatively indicative of the absence below a threshold, the presence above a threshold, or the amount of the second target analyte or of a change in the absence below a threshold, the presence above a threshold, or the amount of the second target analyte.

6. A sensor according to claim 1, comprising a first plurality of first sensing moieties that directly or indirectly bind a first target analyte, a second plurality of second sensing moieties that directly or indirectly bind a second target analyte, and one or more signaling moieties that alone or in combination engender the production of a detectable signal, wherein production of the detectable signal is qualitatively or quantitatively indicative of the absence below a threshold, the presence above a threshold or the amount of the first and the second target analytes in combination or is indicative of a change in the absence below a threshold, the presence above a threshold or the amount of the first and second target analytes in combination.

7. A sensor according to claim 6, wherein the first and second target analytes contribute differently to the combination.

8. A sensor according to claim 1, wherein one or more of the sensing moieties is partly or entirely de novo.

9. A sensor according to claim 1, wherein one or more sensing moieties are partly or entirely the products of in vitro selection or directed evolution.

10. A sensor according to claim 1, wherein the state of one or more sensing moieties is transduced to one or more signaling entities, wherein the sensing moieties and the signaling moieties are in different molecules or multimolecular complexes.

11. A sensor comprised of sensor element comprising a first plurality of sensing moieties and a second plurality of signaling moieties, wherein each sensing moiety has one or more states indicative of a target analyte, the state or a change in the state of one or more of the sensing moieties is transduced to one or more of signaling moieties, one signaling moiety alone or a third plurality of signaling moieties in combination engenders the production of a detectable signal qualitatively or quantitatively indicative of the absence below a threshold, the presence above a threshold or the amount of a target analyte or of a change in the absence below a threshold, the presence above a threshold or the amount of a target analyte, wherein the sensing moiety and the signaling moiety do not naturally occur with one another in the same organism.

12. A sensor according to claim 11, wherein one or more sensing moieties binds one or more target analytes indirectly but not directly.

13. A sensor according to claim 11, wherein one or more sensing moieties is comprised in a sensing module and one or more signaling moieties is comprised in a signaling module, wherein the sensing module can be operatively linked to different signaling modules and the signaling molecule can be operatively linked to different sensing modules.

14. A sensor according to claim 11, wherein production of the detectable signal engenders changes in the sensor element whereby production of the detectable signal is terminated and a change in binding of the one or more target analytes to one or more sensing moieties is required to again engender production of a detectable signal.

15. A sensor according to claim 11, comprising a first plurality of sensing moieties that directly or indirectly bind a first target analyte and a second plurality of sensing moieties that directly or indirectly bind a second target analyte, further comprising one or more first signaling moieties that engender a first detectable signal and one or more second signaling moieties that engender a second detectable signal distinguishable from the first detectable signal, wherein the first detectable signal is qualitatively or quantitatively indicative of the absence below a threshold, the presence above a threshold, or the amount of the first target analyte or of a change in the absence below a threshold, the presence above a threshold, or the amount of the first target analyte and the second detectable signal is qualitatively or quantitatively indicative of the absence below a threshold, the presence above a threshold, or the amount of the second target analyte or of a change in the absence below a threshold, the presence above a threshold, or the amount of the second target analyte.

16. A sensor according to claim 11, comprising a first plurality of first sensing moieties that directly or indirectly bind a first target analyte, a second plurality of second sensing moieties that directly or indirectly bind to a second target analyte, and one or more signaling moieties that engender the production of a detectable signal, wherein production of the detectable signal is qualitatively or quantitatively indicative of the absence below a threshold, the presence above a threshold or the amount of the first and the second target analytes in combination or is indicative of a change in the absence below a threshold, the presence above a threshold or the amount of the first and second target analytes in combination.

17. A sensor according to claim 16, wherein the first and second target analytes contribute differently to the combination.

18. A sensor according to claim 11, wherein the sensing moiety is derived from the antigen binding site of an antibody, the binding site of a bacterial chemosensory receptor, or the ligand-binding site of a ligand binding receptor.

19. A sensor according to claim 11, wherein one or more sensing moieties are partly or entirely the products of in vitro selection or directed evolution.

20. A sensor according to claim 11, wherein the state of one or more sensing moieties is transduced to one or more signaling entities, wherein the sensing moieties and the signaling moieties are in different molecules or multimolecular complexes.

21. A sensor comprised of sensor element comprising a first plurality of sensing moieties and a second plurality of signaling moieties, wherein each sensing moiety has one or more states indicative of a target analyte, the state or a change in the state of one or more of the sensing moieties is transduced to one or more of signaling moieties, one signaling moiety alone or a third plurality of signaling moieties in combination engenders the production of a detectable signal qualitatively or quantitatively indicative of the absence below a threshold, the presence above a threshold or the amount of a target analyte, or of a change in the absence below a threshold, the presence above a threshold or the amount of a target analyte, wherein the sensing moieties are comprised of one or more polypeptides, and one or more regions of the one or more polypeptides has a de novo polypeptide sequence.

22. A sensor according to claim 21, wherein one or more sensing moieties binds one or more target analytes indirectly but not directly.

23. A sensor according to claim 21, wherein one or more sensing moieties is comprised in a sensing module and one or more signaling moieties is comprised in a signaling module, wherein the sensing module can be operatively linked to different signaling modules and the signaling molecule can be operatively linked to different sensing modules.

24. A sensor according to claim 21, wherein production of the detectable signal engenders changes in the sensor element whereby production of the detectable signal is terminated and a change in binding of the one or more target analytes to one or more sensing moieties is required to again engender production of a detectable signal.

25. A sensor according to claim 21, comprising a first plurality of sensing moieties that directly or indirectly bind a first target analyte and a second plurality of sensing moieties that directly or indirectly bind a second target analyte, further comprising one or more first signaling moieties that alone or in combination engender the production of a first detectable signal and one or more second signaling moieties that alone or in combination engender the production of a second detectable signal distinguishable from the first detectable signal, wherein the first detectable signal is qualitatively or quantitatively indicative of the absence below a threshold, the presence above a threshold, or the amount of the first target analyte or of a change in the absence below a threshold, the presence above a threshold or the amount of the first target analyte and the second detectable signal is qualitatively or quantitatively indicative of the absence below a threshold, the presence above a threshold, or the amount of the second target analyte or of a change in the absence below a threshold, the presence above a threshold, or the amount of the second target analyte.

26. A sensor according to claim 21, comprising a first plurality of first sensing moieties that directly or indirectly bind a first target analyte, a second plurality of second sensing moieties that directly or indirectly bind to a second target analyte, and one or more signaling moieties that engender the production of a detectable signal, wherein production of the detectable signal is qualitatively or quantitatively indicative of the absence below a threshold, the presence above a threshold or the amount of the first and the second target analytes in combination or is indicative of a change in the absence below a threshold, the presence above a threshold or the amount of the first and second target analytes in combination.

27. A sensor according to claim 26, wherein the first and second target analytes contribute differently to the combination.

28. A sensor according to claim 21, wherein the sensing moiety is derived partly or entirely from the antigen binding site of an antibody, the binding site of a bacterial chemosensory receptor, or the ligand-binding site of a ligand binding receptor.

29. A sensor according to claim 21, wherein one or more sensing moieties are partly or entirely the products of in vitro selection or directed evolution.

30. A sensor according to claim 21, wherein the state of one or more sensing moieties is transduced to one or more signaling entities, wherein the sensing moieties and the signaling moieties are in different molecules or multimolecular complexes.

31. A sensor according to claim 21, wherein said de novo polypeptide sequence is a de novo designed binary patterned sequence.

32. A sensor according to claim 21, wherein the sensing moiety comprises a four helix bundle receptor protein binding domain.

33. A sensor according to claim 29, wherein the sensing moiety comprises a four helix bundle receptor protein binding domain.

34. A sensor according to claim 30, wherein the sensing moiety comprises a four helix bundle receptor protein binding domain.

35. A sensor according to claim 21, wherein the sensing moieties and the signaling moieties are disposed operationally in a membrane.

36. A sensor according to claim 21, wherein the sensor element is comprised in cell.

37. A sensor according to claim 36, wherein the cell is a bacterial cell.

38. A sensor according to claim 37, wherein the bacterial cell is a chemosensory cell.

39. A sensor according to claim 38, wherein the chemosensory cell comprises methyaccepting chemosensory receptors.

40. A sensor according to claim 21, wherein the de novo polypeptide sequence is comprised in the ligand binding domain of a bacterial methyaccepting chemosensory receptor.

41. A sensor according to claim 40, wherein the signaling moiety is a signaling domain of a methyaccepting chemoreceptor.

42. A sensor according to claim 40, wherein a de novo polypeptide sequence is a de novo designed binary patterned sequence.

43. A sensor according to claim 40, wherein a one or more polypeptides comprises a region that is product of in vitro selection or directed evolution or both of a polypeptide comprising a region of de novo polypeptide sequence.

44. A sensor according to claim 43, wherein the de novo polypeptide sequence is the product of directed evolution of a de novo designed binary patterned sequence.

45. A sensor according to claim 40, wherein one or more sensing moieties binds one or more target analytes indirectly but not directly.

46. A sensor according to claim 40, wherein one or more sensing moieties is comprised in a sensing module and one or more signaling moieties is comprised in a signaling module, wherein the sensing module can be operatively linked to different signaling modules and the signaling molecule can be operatively linked to different sensing modules.

47. A sensor according to claim 40, wherein production of the detectable signal engenders changes in the sensor element whereby production of the detectable signal is terminated and a change in binding of the one or more target analytes to one or more sensing moieties is required to again engender production of a detectable signal.

48. A sensor according to claim 40, comprising a first plurality of sensing moieties that directly or indirectly bind a first target analyte and a second plurality of sensing moieties that directly or indirectly bind a second target analyte, further comprising one or more first signaling moieties that engender a first detectable signal and one or more second signaling moieties that engender a second detectable signal distinguishable from the first detectable signal, wherein the first detectable signal is qualitatively or quantitatively indicative of the absence below a threshold, the presence above a threshold, or the amount of the first target analyte or of a change in the absence below a threshold, the presence above a threshold or the amount of the first target analyte and the second detectable signal is qualitatively or quantitatively indicative of the absence below a threshold, the presence above a threshold, or the amount of the second target analyte or of a change in the absence below a threshold, the presence above a threshold, or the amount of the second target analyte.

49. A sensor according to claim 40, comprising a first plurality of first sensing moieties that directly or indirectly bind a first target analyte, a second plurality of second sensing moieties that directly or indirectly bind to a second target analyte, and one or more signaling moieties that engender the production of a detectable signal, wherein production of the detectable signal is qualitatively or quantitatively indicative of the absence below a threshold, the presence above a threshold or the amount of the first and the second target analytes in combination or is indicative of a change in the absence below a threshold, the presence above a threshold or the amount of the first and second target analytes in combination.

50. A sensor according to claim 49, wherein the first and second target analytes contribute differently to the combination.

51. A sensor according to claim 21, wherein the sensing moiety is derived partly or entirely from the antigen binding site of an antibody, the binding site of a bacterial chemosensory receptor, or the ligand-binding site of a ligand binding receptor.

52. A sensor according to claim 21, wherein one or more sensing moieties are partly or entirely the products of in vitro selection or directed evolution.

53. A sensor according to claim 21, wherein the state of one or more sensing moieties is transduced to one or more signaling entities, wherein the sensing moieties and the signaling moieties are in different molecules or multimolecular complexes.

54. A sensor according to claim 1, wherein the detectable signal is fluorescence, chemiluminescence, phosphorescence or optical absorbence, or a change in fluorescence, chemiluminescence, phosphorescence or optical absorbence.

55. A sensor according to claim 11, wherein the detectable signal is fluorescence, chemiluminescence, phosphorescence or optical absorbence. or a change in fluorescence, chemiluminescence, phosphorescence or optical absorbence.

56. A sensor according to claim 21, wherein the detectable signal is fluorescence, chemiluminescence, phosphorescence or optical absorbence. or a change in fluorescence, chemiluminescence, phosphorescence or optical absorbence.

57. A sensor according to claim 40, wherein the detectable signal is fluorescence, chemiluminescence, phosphorescence or optical absorbence. or a change in fluorescence, chemiluminescence, phosphorescence or optical absorbence.

58. A sensor according to claim 57, wherein the detectable signal is a change in the fluorescence intensity of an acceptor fluorophore of a donor-acceptor fluorescence energy transfer pair.

59. A method for detecting a target analyte comprising exposing a composition in which the target analyte is to be detected to a sensor that produces a detectable signal indicative of the absence below a threshold, the presence above a threshold, the amount or a change in the absence below a threshold, the presence above a threshold or the amount of the target analyte, and determining the detectable signal or the absence thereof, wherein the sensor is comprised of a sensor element comprising one or more sensing moieties and one or more signaling moieties, wherein each sensing moieties has one or more states indicative of a target analyte, each signaling moiety alone or in combination engenders the production of a detectable signal, the state or a change in the state of one or more signaling moieties is transduced to one or more signaling moieties whereby the one or more signaling entities alone or in combination engender the production of a detectable signal qualitatively or quantitatively indicative of the absence below a threshold, the presence above a threshold or the amount of a target analyte or of a change in the absence below a threshold, the presence above a threshold or the amount of a target analyte, and one or the other or both the sensing moiety and the signaling moiety is not a naturally occurring entity.

60. A method for detecting a target analyte comprising exposing a composition in which the target analyte is to be detected to a sensor that produces a detectable signal indicative of the absence below a threshold, the presence above a threshold or the amount, or a change in the absence below a threshold, the presence above a threshold or the amount of the target analyte, and determining the detectable signal or the absence thereof, wherein the sensor is comprised of a sensor element comprising one or more sensing moieties and one or more signaling moieties, wherein each sensing moieties has one or more states indicative of a target analyte, each signaling moiety alone or in combination engenders the production of a detectable signal, the state or a change in the state of one or more signaling moieties is transduced to one or more signaling moieties whereby the one or more signaling entities alone or in combination engender the production of a detectable signal qualitatively or quantitatively indicative of the absence below a threshold, the presence above a threshold or the amount of a target analyte or of a change in the absence below a threshold, the presence above a threshold or the amount of a target analyte, wherein the sensing moieties are comprised of one or more polypeptides, and one or more regions of one or more of the polypeptides has a de novo polypeptide sequence.

61. A bacterial cell comprising a first plurality of sensing moieties and a second plurality of signaling moieties, wherein each sensing moiety has one or more states indicative of a target analyte, the state or a change in the state of one or more of the signaling moieties is transduced to one or more of signaling moieties, one signaling moiety alone or a third plurality of signaling moieties in combination engenders the production of a detectable signal qualitatively or quantitatively indicative of the absence below a threshold, the presence above a threshold or the amount of a target analyte, or of a change in the absence below a threshold, the presence above a threshold or the amount of a target analyte, wherein the sensing moieties are comprised of one or more polypeptides, and one or more regions of the polypeptides has a de novo polypeptide sequence.

62. A method for making a sensor, comprising preparing sensor elements from a bacterial cells culture and incorporating said sensor elements operatively into a sensor, wherein the sensor elements comprise a first plurality of sensing moieties and a second plurality of signaling moieties, wherein each sensing moiety has one or more states indicative of a target analyte, the state or a change in the state of one or more of the signaling moieties is transduced to one or more of signaling moieties, one signaling moiety alone or a third plurality of signaling moieties in combination engenders the production of a detectable signal qualitatively or quantitatively indicative of the absence below a threshold, the presence above a threshold or the amount of a target analyte, or of a change in the absence below a threshold, the presence above a threshold or the amount of a target analyte, wherein one or more sensing moieties is comprised of one or more polypeptides, and one or more regions of one or more of the polypeptides has a de novo polypeptide sequence.
